(19) United States
(12) Patent Application Publication
(10) Pub. No.: US 2022/0193103 A1
(43) Pub. Date: Jun. 23, 2022

(54) NANO-PUERARIN REGULATES TUMOR MICROENVIRONMENT AND FACILITATES CHEMO- AND IMMUNOTHERAPY IN MURINE TRIPLE NEGATIVE BREAST CANCER MODEL

(71) Applicant: The University of North Carolina at Chapel Hill, Chapel Hill, NC (US)

(72) Inventors: Leaf Huang, Chapel Hill, NC (US); Huan Xu, Chapel Hill, NC (US)

(21) Appl. No.: 17/605,377

(22) PCT Filed: Apr. 23, 2020
(86) PCT No.: PCT/US2020/029453

§ 371 Date: Oct. 21, 2021

Related U.S. Application Data
(60) Provisional application No. 62/837,538, filed on Apr. 23, 2019.

(51) Int. Cl. A61K 31/7048; A61K 9/107; A61K 47/24; A61K 47/18; A61K 31/337; A61K 39/395; A61P 35/00

(52) U.S. Cl. CPC A61K 31/7048; A61K 9/1075; A61K 47/24; A61P 35/00; A61K 31/337; A61K 39/3955; A61K 47/18

(57) ABSTRACT

Disclosed are nanoemulsions comprising puerarin and methods of their use in treating cancer, including breast cancer and melanoma. The presently disclosed puerarin-containing nanoemulsions regulate the tumor microenvironment and importantly de-activate tumor associated fibroblasts (TAFs) rather than killing them. The presently disclosed methods can be used in combination with chemotherapy, e.g., polymer formulations of paclitaxel, or PD-L1 blockade therapy to treat cancer.

### FEDERALLY SPONSORED RESEARCH OR DEVELOPMENT

This invention was made with government support under Grant Number CA198999 awarded by the National Institutes of Health. The government has certain rights in the invention.

### BACKGROUND

Most solid tumors contain reactive stromal cells, including tumor-associated fibroblasts (TAFs) and immune cells, vasculature, and extracellular matrix (ECM). As the pivotal effector cells mediating desmoplasia, TAFs are indispensable for the tumor progression in these solid tumors. These highly proliferative TAFs can promote tumor growth through the production of a variety of growth factors. They also are responsible for the recruitment of immunosuppressive cells through the secretion of cytokines and chemokines to protect tumor cells from immune surveillance. Further, the dense ECM produced by TAFs creates high interstitial fluid pressure, which serves as a physical barrier for both drug delivery and cytotoxic T cell penetration. The past five years have witnessed accelerating progress in immune checkpoint blockade therapy for a few types of solid tumors with a high mutational burden. A strong association of transforming growth factor-β (TGF-(β) signaling, a hallmark of TAFs activation, with the compromised response to PD-L1 blockade has previously been demonstrated even in the neoantigen-rich tumor. For instance, PD-1/PD-L1 checkpoint blockers have durable response rate as high as 40% in melanoma, which nevertheless is a typical type of solid tumor rarely containing dense fibrous stroma.

In contrast, triple negative breast cancer (TNBC), which contains the highest mutational frequency of breast cancer subtypes and high PD-L1 expression, but characteristic of geographical or central tumor fibrosis only has up to 20% response to PD-L1 blockade. This relatively ineffectiveness of PD-L1 blockade therapy might be attributed by the abundance of TAFs in TNBC. Therefore, desmoplasia depleting agents have a great potential to facilitate both chemo- and immunotherapy via tumor microenvironment (TME) remodulation. Previous studies have shown that cisplatin, a chemotherapeutic drug, can cause damage to TAFs and inhibit the growth of tumors, however, it correspondingly leads to an increase in Wnt16 in TAFs. Wnt16 is attributed to increase tumor cell resistance and stroma reconstruction.

### SUMMARY

In some aspects, the presently disclosed subject matter provides a nanoemulsion comprising puerarin, or a derivative thereof, for use in treating cancer. In certain aspects, the nanoemulsion comprises lecithin. In more certain aspects, the nanoemulsion further comprises a targeting ligand. In particular aspects, the targeting ligand is aminoethylanisamide (AEAA).

In other aspects, the presently disclosed subject matter provides a method for treating a cancer in a subject in need of treatment thereof, the method comprising administering a therapeutically effective amount of a the presently disclosed nanoemulsion comprising puerarin to the subject to treat the cancer. In certain aspects, the method further comprises treatment with one or more therapeutic agents in combination with the presently disclosed nanoemulsion. In some particular aspects, the one or more therapeutic agents comprises one or more chemotherapeutic agents. In more particular aspects, the one or more chemotherapeutic agents comprises paclitaxel, e.g., a polymer nanoformulation of paclitaxel. In other aspects, the method further comprises a PD-L1 blockade therapy.

In such aspects, the PD-L1 blockade therapy comprises administering α-PD-L1 to the subject in combination with the presently disclosed nanoemulsion.

In some aspects, the presently disclosed methods of treating a cancer include remodeling of a microenvironment of a tumor comprising the cancer. In certain aspects, the method includes deactivating one or more tumor associated fibroblasts

(TAFs). In particular aspects the cancer is selected from breast cancer and melanoma. In yet more particular aspects, the breast cancer comprises triple negative breast cancer.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

The patent or application file contains at least one drawing executed in color. Copies of this patent or patent application publication with color drawings will be provided by the Office upon request and payment of the necessary fee. Having thus described the presently disclosed subject matter in general terms, reference will now be made to the accompanying Figures, which are not necessarily drawn to scale, and wherein:

FIG. 1 is a schematic illustrating tumor microenvironment (TME) remodulation by targeted puerarin delivery;

FIG. 2A, FIG. 2B, FIG. 2C, FIG. 2D, FIG. 2E, FIG. 2F, and FIG. 2G illustrate screening of traditional Chinese medicines (TCMs) on inhibiting reactive oxygen species (ROS) production and characterization of a puerarin nanoemulsion, referred to herein as “nanoPue.” (FIG. 2A) Effects of selected TCMs on ROS inhibition in TGF-β activated NIH3T3 cells. (1) Astragalus total saponins (2) Matrine, (3) Panax notoginseng saponins R1, (4) Puerarin, (5) Jujuboside, (6) Quercetin, (7)

Astragaloside IV, (8) Emodin, (9) Hydroxysafflor yellow A, (10) Tanshinone IIA (n=6). Concentrations of all TCMs were 15 μg/mL. (FIG. 2B) Dynamic light scattering measurements of particle size and distribution of nanoPue. (FIG. 2C) Zeta potential of nanoPue. (FIG. 2D) TEM image of nanoPue. Scale bar represents 200 nm. (FIG. 2E) Appearance of nanoPue. (FIG. 2F) Effects of different concentrations of puerarin and nanoPue on ROS inhibition in TGF-β activated NIH3T3 cells (n=6). (FIG. 2G) The chemical structure of puerarin;

FIG. 3A, FIG. 3B, FIG. 3C, and FIG. 3D show (FIG. 3A) Stability of nanoPue (n=3). Changes in particle sizes and EE % when nanoPue were stored for different times at 4° C. (FIG. 3B) Drug release of puerarin suspension and nanoPue in PBS (pH 7.4) at 37° C. (n=3). (FIG. 3C) Mean plasma concentration-time curves of free drug and nanoPue after intravenous injection in mice (n=7). (FIG. 3D) Mean pharmacokinetic parameters of puerarin suspension and nanoPue in mouse (n=7).** P<0.01, ***P<0.001;

FIG. 4A shows the effect of puerarin and nanoPue on 3T3 cell viability. The cell viability was measured by MTT assay. (n=6) FIG. 4B shows a hemolytic assay of nanoPue at various concentrations. NG represents negative control group, PG represents positive control group. Data are represented as the mean±S.D. (n=3);

FIG. 5A, FIG. 5B, FIG. 5C, FIG. 5D, FIG. 5E, FIG. 5F, FIG. 5G, FIG. 5H, FIG. 5I, and FIG. 5J demonstrate the toxicity evaluation of nanoPue (FIG. 5A-FIG. 5D) and attenuated 4T1 desmoplastic reaction by the nanoPue treatment (FIG. 5E-FIG. 5I). (FIG. 5A) PBS, blank emulsion and nanoPue treatment scheme. (FIG. 5B) Serum ALP, ALT, AST, BUN, and creatinine levels (n=4). (FIG. 5C) H&E staining of major drug accumulating organs after 6 injections of different treatments (n=5). Scale bar represents 50 μm. (FIG. 5D) Mice body weight changes under tumor inhibition study (n=7). (FIG. 5E) The tumor weight after 6 injections of different treatment. (n=7). (FIG. 5F) Confocal microscopy identifying α-SMA. The quantification results expressed as the percentage of total cell number (n=5). Scale bar represents 20 μm. (FIG. 5G) Masson's trichrome staining and quantification of collagen deposition expressed as the percentage of total cell number (n=5). Scale bar represents 50 μm. (FIG. 5H) Immune cells obtained from tumor analyzed by using flow cytometry (n=4). α-SMA in the 4T1 tumors after various treatments. (FIG. 5I) RT-PCR analysis of TGF-β, FGF-2, TNF-α and PDGF-B expression in the tumor tissue after different treatment (n=6). (FIG. 5J) Western blot analysis of HIF-1α, NOX4, p-SMAD2, p-SMAD3, α-SMA and GAPDH expression in the 4T1 tumor after different treatments. Quantification of protein was obtained via with ImageJ analysis, and normalized with GAPDH (n=4).*P<0.05, ** P<0.01, *** P<0.001, **** P<0.0001;

FIG. 6 shows confocal microscopy images identifying α-SMA and DiD in 4T1 tumor. The quantification results expressed as the percentage of total cell number (n=5). Scale bar represents 20 μm. *** P<0.001, **** P<0.0001;

FIG. 7A, FIG. 7B, FIG. 7C, and FIG. 7D demonstrate the concomitant effect of nanoPue treatment on the behaviors of nanoDiD within tumors. (FIG. 7A) nanoPue and nanoDiD treatment scheme. (FIG. 7B) Images and quantitative results of the nanoDiD in the mice and tumors at 24 h after test particle injection (n=5). (FIG. 7C) The tumors were excised and sectioned into 10 μm thick slices and observed by laser scanning confocal microscope. The particles are shown in green (DiD) and the blood vessels are shown in red (CD31). Scale bar represents 50 μm. The green fluorescence intensity profile as a function of distance from blood vessels (0-25 μm) in a representative region (indicated by the yellow rectangle) was plotted by using software ImageJ (FIG. 7D). * P<0.05, ** P<0.01, *** P<0.001, ns: not significant;

FIG. 8A and FIG. 8B are laser scanning confocal microscope images identifying α-SMA and CD31 in BPD6 (FIG. 8A) and 4T1 (FIG. 8B) tumor. The quantification results expressed as the percentage of total cell number (n=5). Scale bar represents 20 μm. ** P<0.01, *** P<0.001, **** P<0.0001; FIG. 9A, FIG. 9B, FIG. 9C, FIG. 9D, and FIG. 9E show the combination of nanoPue and nanoPTX therapy on 4T1 tumor model (n=5). (FIG. 9A) nanoPue and nanoPTX combination treatment scheme. (FIG. 9B) Tumor growth curves of 4T1 tumors in different treated groups. (FIG. 9C) Tumor images and weight at the end of the experiment. (FIG. 9D) TUNEL staining of differently treated 4T1 tumor tissues. TUNEL positive cells were quantified in 3 randomly selected fields per mouse (n=5). (E) Comparison of Ki67 expression of 4T1 tumors in different treatment groups. Scale bar represents 20 μm. Scale bar represents 20 μm. * P<0.05, ** P<0.01, *** P<0.001 and **** P<0.0001;

FIG. 10A, FIG. 10B, FIG. 10C, and FIG. 10D demonstrate the toxicity evaluation of the combined administration of nanoPue and nanoPTX. (FIG. 10A)

Serum ALT, AST, BUN, and creatinine levels. (FIG. 10B) Body weight change. (FIG. 10C) Survival among different treatments (n=5). (FIG. 10D) H&E staining of major drug accumulating organs after different treatments. Scale bar represents 50 μm.* P<0.05, ** P<0.01, ns: not significant;

FIG. 11A and FIG. 11B show nanoPue induced 4T1 tumor immune microenvironment changes (FIG. 11A) RT-PCR analysis of IL-4, IL-6, IL-10, IL-13, CCL2 and CCL5 expression in the tumor tissue after different treatments (n=6). (FIG. 11B) Analysis of CD8⁺, CD4⁺ T cells. MDSC, Tregs, M1/M2 ratios in the 4T1 tumors after various treatments by using flow cytometry (n=4). *P<0.05, **P<0.01, ***P<0.001, ****P<0.0001;

FIG. 12A, FIG. 12B, FIG. 12C, FIG. 12D, and FIG. 12E demonstrate the combination of nanoPue and α-PD-L1 therapy on 4T1 tumor model (n=5). (FIG. 12A) nanoPue and α-PD-1,1 combination treatment scheme. (FIG. 12B) Tumor growth curves of 4L1 tumors in different treatment groups. (FIG. 12C) Tumor weight at the end of the experiment. (FIG. 12D) TUNEL staining of α-PD-L1 and nanoPue combined with α-PD-L1 treated 4T1 tumor tissues. TUNEL positive cells were quantified in 3 randomly selected fields per mouse (n=5), (FIG. 12E) Comparison of Ki67 expression of 4T1 tumors in different treatment groups (n=5). Scale bar represents 20 μm* p<0.05, ** p<0.01, *** p<0.001, **** p<0.0001;

FIG. 13A, FIG. 13B, FIG. 13C, FIG. 13D, and FIG. 13E show the toxicity and survival evaluation of combined administration of nanoPue and α-PD-L1 on 4T1 tumor model. (FIG. 13A) The representative tumor image in different treatment groups. (FIG. 13B) Serum ALT, AST, BUN, and creatinine levels. (n=3) (FIG. 13C) Body weight change. (n=5) (FIG. 13D) Survival among different treatments. (n=5). (FIG. 13E) H&E staining of major drug accumulating organs after different treatments. (n=3) Scale bar represents 50 μm. ns: not significant; and

FIG. 14A and FIG. 14B. Particle size (FIG. 14A) and Zeta-potential (FIG. 14B) of the reconstituted nano-formulation (ZY-010) in 0.9% NaCl solution. The results are based on the concentration of paclitaxel at 100 μg/mL.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. NANO-PUERARIN REGULATES TUMOR MICROENVIRONMENT AND FACILITATES CHEMO-AND IMMUNOTHERAPY IN MURINE TRIPLE NEGATIVE BREAST CANCER MODEL

### A. Nanoemulsions Comprising Puerarin

In some embodiments, the presently disclosed subject matter provides a nanoemulsion comprising puerarin, or a derivative thereof, for use in treating cancer. Puerarin is an isoflavone and is found in a number of plants and herbs, including kudzu. Puerarin has the following chemical structure:

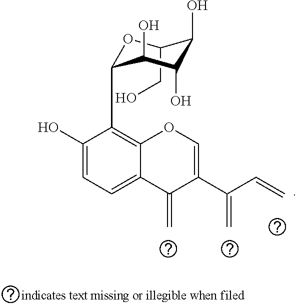

In particular embodiments, the nanoemulsion comprises lecithin. In certain embodiments, the nanoemulsion comprises a targeting ligand. In particular embodiments the targeting ligand is aminoethylanisamide (AEAA).

In certain embodiments, the nanoemulsion comprises spherical particles. In certain embodiments, the spherical particles can have a diameter of less than about 150 nm, including but not limited to about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, and 150 nm. In particular embodiments, the spherical particles have a diameter of between about 100 nm and about 125 nm. In more particular embodiments, the spherical particles have a diameter of about 112 ±5 nm.

In some embodiments, the nanoemulsion has a zeta potential of between about −10 mV to about −2 mV. In certain embodiments, the nanoemulsion has a zeta potential of about −5.3±0.6 mV. In certain embodiments, the nanoemulsion has an encapsulation efficiency of between about 75% and 90% for puerarin, including 75%, 80%, 85%, and 90%. In particular embodiments, the nanoemulsion has an encapsulation efficiency of about 82.4±3.2% for puerarin.

### B. Method for Treating a Cancer

In some embodiments, the presently disclosed subject matter provides a method for treating a cancer in a subject in need of treatment thereof, the method comprising administering a therapeutically effective amount of a presently disclosed nanoemulsion comprising puerarin, or a derivative thereof, to the subject to treat the cancer. As used herein, the term “cancer” refers to or describe the physiological condition in mammals that is typically characterized by unregulated cell growth, As used herein, “cancer cells” or “tumor cells” refer to the cells that are characterized by this unregulated cell growth.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the term “inhibit,” and grammatical derivations thereof, refers to the ability of a presently disclosed compound, e.g., a presently disclosed compound of formula (I), to block, partially block, interfere, decrease, or reduce the growth of bacteria or a bacterial infection. Thus, one of ordinary skill in the art would appreciate that the term “inhibit” encompasses a complete and/or partial decrease in the growth of bacteria or a bacterial infection, e.g., a decrease by at least 10%, in some embodiments, a decrease by at least 20%, 30%, 50%, 75%, 95%, 98%, and up to and including 100%.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In certain embodiments, the presently disclosed method further comprises treatment with one or more therapeutic agents in combination with the presently disclosed nanoemulsion.

The term “combination” is used in its broadest sense and means that a subject is administered at least two agents, more particularly a presently disclosed nanoemulsion comprising puerarin and at least one additional therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the presently disclosed nanoemulsion comprising puerarin described herein can be administered alone or in combination with adjuvants that enhance stability of the nanoemulsion, alone or in combination with one or more antibacterial agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. The timing of administration of a presently disclosed nanoemulsion comprising puerarin and at least one additional therapeutic agent can be varied so long as the beneficial effects of the combination of these agents are achieved. Accordingly, the phrase “in combination with” refers to the administration of a presently disclosed nanoemulsion comprising puerarin and at least one additional therapeutic agent either simultaneously, sequentially, or a combination thereof.

Therefore, a subject administered a combination of a presently disclosed nanoemulsion comprising puerarin and at least one additional therapeutic agent can receive a presently disclosed nanoemulsion comprising puerarin and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the presently disclosed nanoemulsion comprising puerarin and at least one additional therapeutic agent are administered simultaneously, they can be administered to the subject as separate pharmaceutical compositions, each comprising either a presently disclosed nanoemulsion comprising puerarin or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a presently disclosed nanoemulsion comprising puerarin and at least one additional therapeutic agent is greater than the sum of the biological activities of the respective agents when administered individually.

Synergy can be expressed in terms of a combination index (CI, which can be determined, for example, by using the Chou and Talalay method. Zhang et al., 2014; Chou et al., 1984. CI can be calculated by using the following equation (1):

CI=(D)₁/(Dₓ)₁+(D)₂/(Dₓ)₂ (1)

where (D)₁ and (D)₂ are the concentrations for a single drug after combination that inhibits x % of cell growth, and (Dₓ)₁ and (Dₓ)₂ are the concentrations for a single drug alone that inhibits x % of cell growth. CI values more than one demonstrate antagonism and CI values less than one demonstrate synergism of drug combinations.

In general, the lower the CI, the greater the synergy shown by that particular combination. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

In certain embodiments, the one or more therapeutic agents comprises one or more chemotherapeutic agents. As used herein, a “chemotherapeutic agent” is a chemical compound or biologic useful in the treatment of cancer. In embodiments, non-limiting examples of chemotherapeutic agents include alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; ethylenimines and methvlamelainines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®)), CPT-11 (irinotecan, CAMPTOSAR®), acetylcamptothecin, scopolectin, and 9-aminocamptothecin); brostatin; pemetrexed; callystatin; CC-1065 (including its adozelesin, carzelesin and hizelesin synthetic analogues); podophyllotoxin; podophyllinic acid; teniposide; cryptophycins (particularly cryptophycin I and cryptophycin 8); dolastatin; duocarmycin (including the synthetic analogues, KW-2189 and CB 1-TM1); eleutherobin; pancratistatin; TLK-286; CDP323, an oral alpha-4 integrin inhibitor; a sarcodictyin; spongistatin;

nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegall (see, e.g., Nicolaou et ah, Angew. Chem Intl. Ed. Engl., 33: 183-186 (1994)); dynemicin, including dynemicin A; an esperamicin; as well as neocarzino statin chromophore and related chromoprotein enediyne antibiotic chromophores), aclacinomysins, actinomycin, authramycin, azaserine, bleomycins, cactinomyein, carabicin, carminomycin, carzinophilin, chromomycinis, dactinomycin, daunorubicin, detorubicin, 6-diazo-5-oxo-L-norleueine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluoro uracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitahine, and floxuridine; anti-adrenals such as aminoglutethimide, mitotane, trilostane; folic acid replenisher, such as frolinic acid; aceglatone; aldophosphamide glycoside; aminolevulinic acid; eniluracil;

amsacrine; bestrabucil; bisantrene; edatraxate; defofamine; demecolcine; diaziquone; elfomithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g. paclitaxel (TAXOL®), albumin-engineered nanoparticle formulation of paclitaxel (ABRAXANE™), and doxetaxel (TAXOTERE®); chlorambucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylomithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

In certain embodiments, the one or more chemotherapeutic agents comprises paclitaxel. In particular embodiments, the paclitaxel comprises a polymer nanoformulation of paclitaxel.

In other embodiments, the presently disclosed method for treating a cancer further comprises a PD-L1 blockade therapy. In such embodiments, the PD-L1 blockade therapy comprises administering α-PD-L1 to the subject in combination with the presently disclosed nanoemulsion comprising puerarin.

Importantly, the presently disclosed methods remodel of a microenvironment of a tumor comprising the cancer. In particular embodiments, the method for treating cancer includes deactivating one or more tumor associated fibroblasts (TAFs). In yet more particular embodiments, the method for treating of the cancer includes a reduction of α-SMA positive TAFs in one or more tumors comprising the cancer and/or a inhibiting expression of α-SMA in one or more tumors comprising the cancer.

In particular embodiments, method the treating of the cancer includes downregulation of reactive oxygen species (ROS) production in an activated myofibroblast. In certain embodiments, the method reduces deposition of collagen in the extracellular matrix (ECM). In yet more particular embodiments, the method alleviates desmoplasia.

In particular embodiments, the method includes a reduction of intratumoral IL-4, IL-6, IL-10 and IL-13. In other embodiments, the method includes increasing infiltration of CD8⁺ and CD4⁺ T cells into a tumor of the cancer. In yet other embodiments, the treating of the cancer includes one or more of downregulation of CCL2 and CCL5, reducing intratumoral Th2 cytokine levels, reducing Tregs and MDSCs infiltration, promoting M2 macrophage phenotype switch to pro-inflammatory M1, and combinations thereof.

In certain embodiments, the treating of the cancer inhibits one or more profibrogenic cytokines. In particular embodiments, the one or more profibrogenic cytokines are selected from the group consisting of transforming growth factor-β (TGF-(β), fibroblast growth factor (FGF-2), platelet-derived growth factor B (PDGF-B), and tumor necrosis factor (TNF-α). In yet other embodiments, the treating of the cancer downregulated the expression of NOX4, HIF-1α, α-SMA, p-SMAD2 and p-SMAD3 in a tumor comprising the cancer.

In certain embodiments, the treating of the cancer includes increasing an enhanced permeability and retention (EPR) effect of a tumor comprising the cancer. In such embodiments, increasing the enhanced permeability and retention (EPR) effect of a tumor comprising the cancer includes reducing a fibrogenic status of one or more fibroblasts, increasing vessel permeability, and reducing an interstitial fluid pressure of a tumor comprising the cancer.

In certain embodiments, the presently disclosed method reduces metastasis of the cancer. In other embodiments, the method decreases a weight of a tumor comprising the cancer. In yet other embodiments, the method inhibits growth of a tumor comprising the cancer.

In some embodiments, the cancer is selected from breast cancer and melanoma. In particular embodiments, the breast cancer comprises triple negative breast cancer. One of ordinary skill in the art would appreciate that other cancers could be treated by the presently disclosed methods, including, but not limited to, all forms of carcinomas, melanomas, sarcomas, lymphomas and leukemias, including without limitation, bladder carcinoma, brain tumors, breast cancer, cervical cancer, colorectal cancer, esophageal cancer, endometrial cancer, hepatocellular carcinoma, laryngeal cancer, lung cancer, osteosarcoma, ovarian cancer, pancreatic cancer, prostate cancer, renal carcinoma and thyroid cancer, In some embodiments, the cancer to be treated is a metastatic cancer. In particular, the cancer may be resistant to known therapies.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Nano-Puerarin Regulates Tumor Microenvironment and Facilitates Chemo-and Immunotherapy in Murine Triple Negative Breast Cancer Model

### 1. 1 Overview

Tumor associated fibroblasts (TAFs) are key stromal cells mediating the desmoplastic reaction and are partially responsible for the drug-resistance and immunosuppressive microenvironment formation in solid tumors. As shown previously, delivery of genotoxic drugs off-targetedly to kill TAFs results in production of Wnt16, which renders the neighboring tumor cells drug resistant. Miao et al., 2015. The presently disclosed subject matter investigates ways to deactivate, rather than kill, TAFs. Reactive oxygen species (ROS) are the central hub of multiple profibrogenic pathways and indispensable for TAFs activation. Herein, puerarin was identified to effectively downregulate ROS production in the activated myofibroblast. More particularly, a novel puerarin nanoemulsion (referred to herein as “nanoPue”) was developed to improve the solubility and bioavailability of puerarin. NanoPue significantly deactivated the stromal microenvironment (e.g., about a 6-fold reduction of TAFs in nanoPue treated mice compared with the PBS control, P<0.0001) and facilitated the chemotherapy effect of nano-paclitaxel in a desmoplastic triple-negative breast cancer (TNBC) model. Moreover, nanoPue successfully stimulated the immune microenvironment, removed the physical barrier for a 2-fold increase of cytotoxic T cell penetration, and therefore improved the effect of the PD-L1 blockade therapy in the TNBC model.

### 1.2 Introduction

### 1.2.1 Background

Most solid tumors contain reactive stromal cells, including tumor-associated fibroblasts (TAFs) and immune cells, vasculature, and extracellular matrix (ECM). As the pivotal effector cells mediating desmoplasia, TAFs are indispensable for the tumor progression in these solid tumors. These highly proliferative TAFs can promote tumor growth through the production of a variety of growth factors. Bremnes et al., 2011. They also are responsible for the recruitment of immunosuppressive cells through the secretion of cytokines and chemokines to protect tumor cells from immune surveillance. Valkenburg et al., 2018. Furthermore, the dense ECM produced by TAFs creates high interstitial fluid pressure, which serves as a physical barrier for both drug delivery and cytotoxic T cell penetration. Zhang et al., 2016. The past five years have witnessed accelerating progress in immune checkpoint blockade therapy for a few types of solid tumors with a high mutational burden. A recent study, however, demonstrated a strong association of transforming growth factor-β (TGF-(β) signaling, a hallmark of TAFs activation, with the compromised response to PD-L1 blockade even in the neoantigen-rich tumor. Mariathasan et al., 2018. For instance, PD-1/PD-L1 checkpoint blockers have durable response rate as high as 40% in melanoma, which nevertheless is a typical type of solid tumor rarely containing dense fibrous stroma. Wiesner et al., 2015; Zhao and Subramanian, 2017.

In contrast, triple negative breast cancer (TNBC), which contains the highest mutational frequency of breast cancer subtypes and high PD-L1 expression, but characteristic of geographical or central tumor fibrosis, Carey et al., 2010, only has up to 20% response to PD-L1 blockade. Denkert et al., 2017. This relatively ineffectiveness of PD-L1 blockade therapy might be attributed by the abundance of TAFs in TNBC. Therefore, desmoplasia depleting agents have a great potential to facilitate both chemo- and immunotherapy via tumor microenvironment (TME) remodulation.

Previous studies have shown that cisplatin, a chemotherapeutic drug, can cause damage to TAFs and inhibit the growth of tumors, however, it correspondingly leads to an increase in Wnt16 in TAFs. Wnt16 is attributed to increase tumor cell resistance and stroma reconstruction. Miao et al., 2015.

### 1.2.2 Scope of Work

The presently disclosed subject matter aimed to deactivate TAFs rather than directly damage TAFs. Reactive oxygen species (ROS) is the key downstream mediator of multiple profibrogenic pathways, including TGF-β and platelet-derived growth factor (PDGF), which are two major desmoplasia initiating factors. In turn, increased ROS in the tumor nest can prolong these fibrogenesis signaling and accelerate desmoplasia. Arcucci et al., 2016. Moreover, chronic ROS stress, which is the case in cancers, is related to the impaired function of the effector T cells via the activation of apoptotic pathways of T cells. Yang et al., 2013. As such, ten traditional Chinese medicines (TCMs) that were known to exhibit anti-fibrotic effect based on their capacity of ROS downregulation in the activated fibroblast were screened.

Compared with conventional screening methodology with cytotoxicity as the readout, the current screening strategy avoided the exclusion of drugs (e.g., TCMs) that show weak cytotoxic effect, but can effectively calm down the activated TAFs.

TCMs have evolved over thousands of years with a unique system of theories for medicinal intervention. These natural active ingredients with low toxicity have been increasingly used as an adjuvant therapy to alleviate cancer symptoms in the last decades. Puerarin is an isoflavone derivative isolated from the kudzu root with the capacity of lowering blood pressure, reducing myocardial oxygen consumption, expanding coronary vessels, protecting liver, controlling blood sugar and inhibiting ischemia-reperfusion injury. Bacanli et al., 2018. Particularly, puerarin shows a substantial anti-fibrosis effect in multiple organs including heart, lung, kidney, and liver. Wei et al., 2014; Hou et al., 2014. The presently disclosed subject matter further confirmed that puerarin showed a superior ROS reduction efficiency in the activated NIH3T3 murine fibroblasts. Puerarin's poor water solubility and bioavailability, however, have limited its application as a pharmaceutical agent.

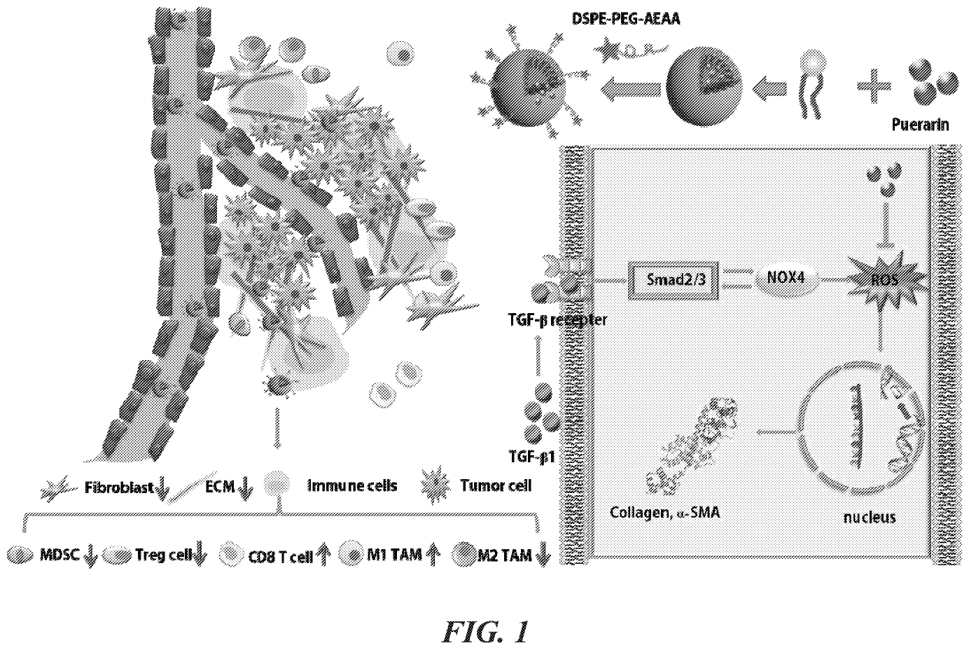

Accordingly, in some embodiments of the presently disclosed subject matter, a novel puerarin nanoemulsion (referred to herein as “nanoPue”) was engineered to improve its pharmacokinetic profile, as well as tumor-specific accumulation. Based on the 4T1 murine TNBC tumor model, nanoPue was confirmed to successfully remodel the tumor stromal microenvironment and dwindle the physical barrier for particle and cell penetration (FIG. 1). Similar results were observed in a murine desmoplastic melanoma model. Further, the combination of nanoPue and paclitaxel (PTX) polymer displayed a synergistic anti-tumor effect with negligible side effects.

In addition, the enhanced immune microenvironment by nanoPue significantly improved the therapeutic efficacy of PD-L1 monoclonal antibody (α-PD-L1). These findings suggest nanoPue can be used as an adjuvant therapy to enhance chemo- and checkpoint blockade immunotherapy in highly desmoplastic solid tumors.

### 1.3 Results and Discussion

### 1.3.1. Inhibitory Effect of Puerarin on ROS Generation and Construction of NanoPue.

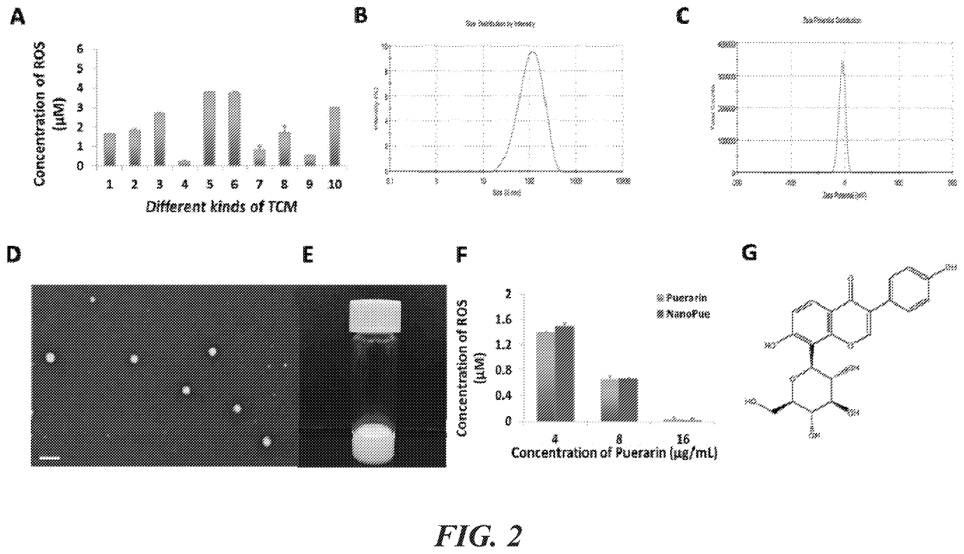

ROS plays a very important role in the desmoplastic reaction. A small amount of ROS has an immune defense effect, however, excessive production of ROS can cause oxidative stress, activation of ERK½ pathway, differentiation of myofibroblasts and abnormal synthesis of ECM protein, which leads to fibrosis and tumor formation (FIG. 1). Meitzler et al., 2014; Son et al., 2017. In the presently disclosed subject matter, ROS assay was used to investigate and compare the inhibitory effects on ROS generation of ten natural compounds. As shown in FIG. 2A, compared with other natural compounds, puerarin significantly reduced the amount of

ROS in TGF-β activated NIH3T3 cells. Thus, further studies focused on this puerarin.

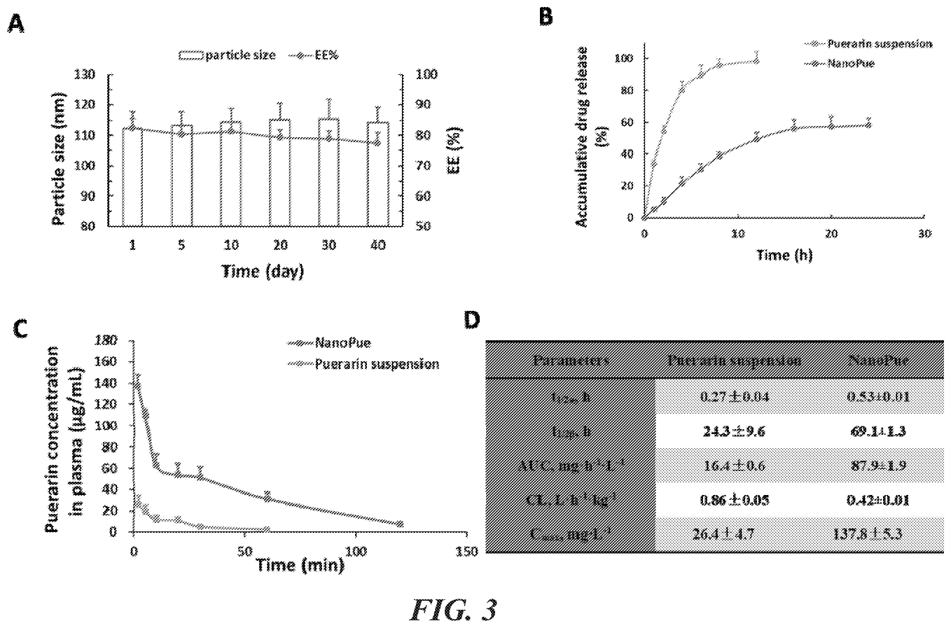

Puerarin is poorly soluble. Its low bioavailability and acute intravascular hemolysis further limit its pharmaceutical application. Wei and Zhang, 2013; Quan and Xu, 2007; Chung et al., 2008. Nanoemulsion is a colloidal particulate system allowing for the improvement of drug solubilization and therapeutic efficacy enhancement. In nanoemulsion-based delivery system, the combination of surfactants with oils offers a superior advantage over a cosolvent system or other nanocarriers in terms of safety profile and drug-loading capacity for hydrophobic compound. To avoid the toxicity of traditional small molecular surfactants, the biocompatible lecithin from soybean was chosen as the principle emulsifier for the preparation of nanoemulsion to carry puerarin (NanoPue). To achieve TAFs targeting ability, NanoPue was surface modified with the targeting ligand, aminoethyl anisamide (AEAA). AEAA is a potent ligand for the sigma receptor, which is overexpressed on most cancer cells and TAFs. Banerjee et al., 2004; Goodwin and Huang, 2016. Recent studies have shown the up-regulation of sigma receptor on TAFs, which is related to the increase of α-SMA. van Waarde et al., 2015. Since most of the desmoplastic tumors have their vessels located in or near the stroma, which is enriched with TAFs, AEAA-modified nanoparticles accumulate mostly in TAFs rather than in tumor cells. Miao et al., 2016. This phenomenon was observed in several different desmoplastic tumors and was called “binding site barrier.” Miao et al., 2016. The encapsulation efficiency (EE) of nanoPue was 82.4±3.2%. The average particle size and zeta potential of nanoPue was 112±5 nm and −5.3±0.6 mV, respectively (FIG. 2B and FIG. 2C), according to dynamic light scattering (DLS) analysis. The nanoemulsion appeared milky white and was remarkably stable under 4° C. with little free drug leakage and unchanged particle size within 40 days (FIG. 3A). The transmission electron microscopy (TEM) image confirmed the size of nanoPue and indicated the spherical shape and homogenous distribution (FIG. 2D). Compared with puerarin suspension (with glycerol as the cosolvent), nanoPue exhibited improved release stability and had an accumulative drug release that only reached 58% within 24 h in PBS (pH 7.4) (FIG. 3B). The pharmacokinetics of nanoPue and puerarin suspension was further investigated in mice. NanoPue increased the half-life (tva) and the area under the curve (AUC) of puerarin by 2-fold and 5-fold, respectively (FIG. 3C and FIG. 3D), which allows for the prolonged therapeutic effect of puerarin. Due to the poor solubility and pharmacokinetic profile of free puerarin, the following in vivo studies only focused on nanoPue.

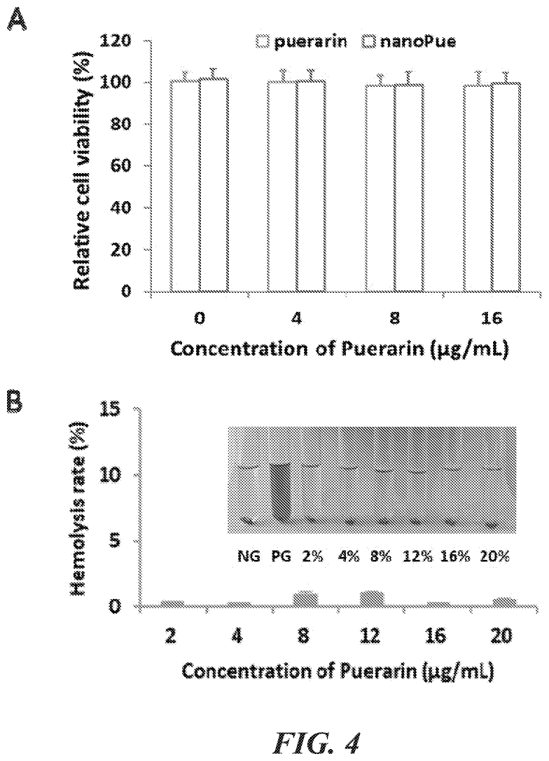

The ROS inhibition activity of nanoPue was compared with that of Puerarin. Both displayed a concentration-dependent ROS downregulation pattern (FIG. 2F). Thus, the emulsification process in manufacturing nanoPue did not affect the pharmaceutical activity of the active ingredient. Different concentrations of puerarin and nanoPue has no cytotoxicity in NIH3T3 cells within 48 hours (FIG. 4A), suggesting that the effect of puerarin on ROS production was not due to its cytotoxicity. Indeed, puerarin has been reported to significantly improve the activity of superoxide dismutase, block the automatic oxidation of lipids, and effectively scavenge oxygen free radicals. The potent antioxidant effect is associated with the presence of 3′-hydroxyl group of puerarin, which plays an important role in the clearance of ONOO- and total ROS (FIG. 2G). Jin et al., 2012; Liu et al., 2013; and

Zhang et al., 2006. In addition, as shown in FIG. 4B, nanoPue at different concentrations showed a negligible hemolytic effect on the red blood cells (RBCs) (hemolyzed erythrocytes <1%) compared with deionized water.

### 1.3.2. NanoPue is Safe and Attenuates Desmoplastic Reaction and Remodels Stromal TME.

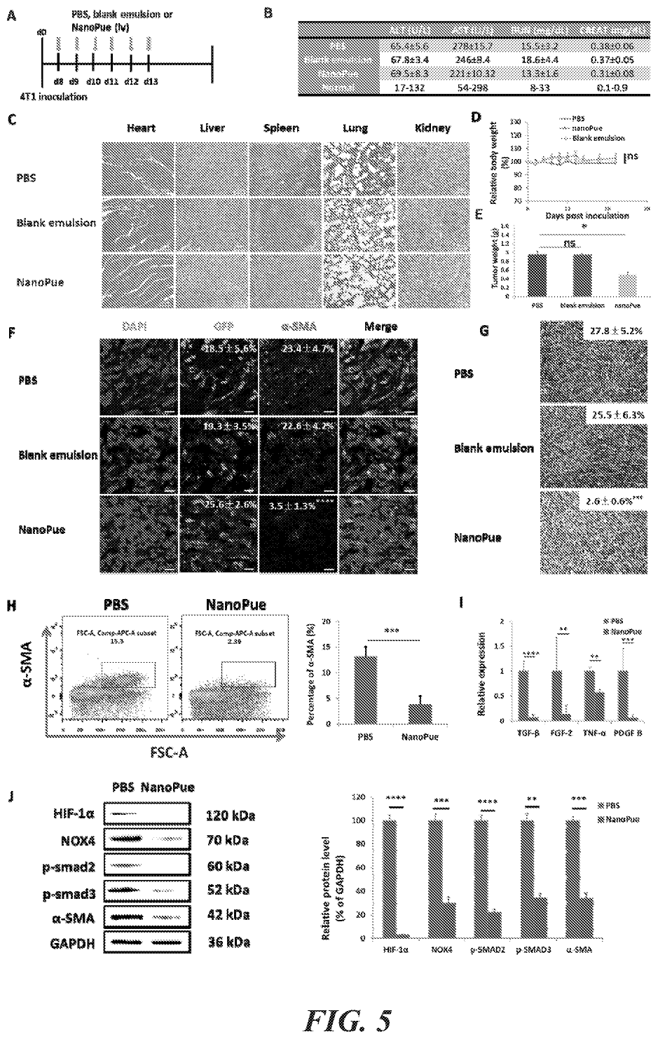

NanoPue was daily injected into Balb/C mice bearing the orthotopic 4T1 breast cancer for 6 days (FIG. 5A) to evaluate the safety profile of the formulation. No significant differences in alanine aminotransferase (ALT), aspartate aminotransferase (AST), blood urea nitrogen (BUN), and creatinine levels were observed after the administration of PBS, blank emulsion and nanoPue (FIG. 5B), which indicated continuous iv injection of nanoPue did not cause renal or hepatic dysfunctions. No significant weight loss occurred during the treatment for all mice (FIG. 5C). To evaluate the toxic effects of nanoPue on the major organs, H&E staining of the major organs were characterized (FIG. 5D). No inflammation or necrosis was found in the heart, liver, spleen and kidney of mice treated with blank emulsion and nanoPue. Lung, however, seems to be the major metastatic site in the PBS and blank emulsion group. The nanoPue seems to reduce lung metastasis to some extent (FIG. 5D). The results of safety evaluation confirmed that the nanoPue did not cause toxicity or side effects in vivo.

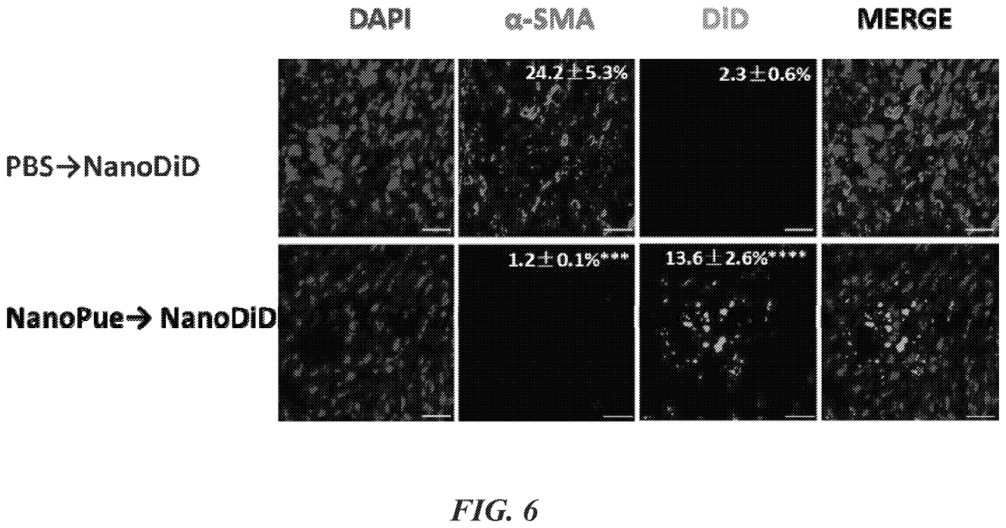

After 6 consecutive daily injections of different formulations, the mice were sacrificed on day 19. The tumor was collected and weighed, followed by the characterization of the parameters of TME, which is orchestrated by various cells and cytokines. As shown in FIG. 5E, nanoPue significantly decreased the tumor weight compared with PBS or blank emulsion group (P<0.05), although with only modest growth inhibition efficiency. Indeed, the major effect of nanoPue was TME remodeling. TAFs, the pivotal effector cells mediating the stromal TME, are activated myofibroblasts most reliably characterized by alpha smooth muscle actin (α-SMA). Clement et al., 2015. FIG. 5F, FIG. 5H and FIG. SI demonstrated significant reduction of α-SMA positive TAFs in tumors of nanoPue treated animals compared with those treated with either PBS or blank emulsion. The immunofluorescence staining showed that diffuse intracytoplasmic α-SMA was abundant in PBS and blank emulsion group, while the expression of α-SMA in nanoPue group was very low (22.6-23.4% vs 3.5%, P<0.0001) (FIG. 5F). Flow cytometry analysis further confirmed significantly lower expression of α-SMA in the nanoPue treated tumor vs the PBS group (FIG. 5H). Similar results were obtained in BPD6 melanoma bearing mice (FIG. 6A).

Collagen is the main ECM composition and serves as another important indicator of the severity of the desmoplastic reaction. Masson's trichrome staining indicated abundant collagen deposition in PBS and blank emulsion groups, while that of the nanoPue group was significantly decreased (P<0.001) (FIG. 5G). In summary, nanoPue could alleviate desmoplasia by deactivating TAFs and reducing collagen deposition. To be noted, repeated injections of blank emulsion did not show anti-tumor nor the stroma remodulation effect (FIG. 5E, FIG. 5F, and FIG. 5G). Therefore, in the follow-up study, the PBS group was used as the only control group.

TGF-β is the most potent and ubiquitous profibrogenic cytokine promoting TAFs activation and ECM deposition. Kato et al., 2007; de Souza Oliveira et al., 2017. RT-PCR results showed that TGF-β was more than 10-fold reduced by nanoPue treatment (P<0.0001) (FIG. 50. Other profibrogenic cytokines including fibroblast growth factor (FGF-2), platelet-derived growth factor B (PDGF-B), and tumor necrosis factor (TNF-α), are involved in promoting collagen synthesis and inhibiting extracellular matrix degradation, Selman et al., 2004, also displayed significantly decreased levels compared with those of PBS group (FIG. 50. The mechanism underlying the induction of fibrosis by TGF-β has been studied intensively in the past years. Increasing evidence indicates that ROS plays a central role in the profibrogenic activity of TGF-β. NADPH oxidases (NOX) enzymes are heme-containing proteins with a primary function of transporting electrons from NADPH to oxygen. Therefore, the NOX family has been confirmed to produce the majority of intracellular ROS in various cells. NOX4, in particular, has been identified to be the most widely distributed in nonphagocytic myofibroblasts and could be upregulated in a SMAD⅔ dependent manner. Samarakoon et al., 2013; Michaeloudes et al., 2010; and Veith et al., 2017. In turn, NOX4 activation and increased ROS level promote SMAD⅔ phosphorylation and facilitates the formation of a feed-forward loop in TGF-β/SMAD profibrogenic signaling. Chan et al., 2013. Hypoxia-induced factor 1α (HIF-1α) is a heterodimeric transcription factor serving as the intracellular ROS sensor. Upon the activation via elevated ROS, upregulated HIF-1α enhances transcriptional activities linked to proliferation of myofibroblasts and inhibition of apoptosis. Meanwhile, the overexpressed HIF-1α could further promote intracellular ROS level and upregulate the expression and activity of NOXs. Feng et al., 2016; Garrido-Urbani et al., 2014. Herein, the expression of NOX4, HIF-1α, α-SMA, p-SMAD2 and p-SMAD3 in the tumor tissue were dramatically down-regulated by the nanoPue treatment according to the western blot analysis (FIG. 5J), confirming that nanoPue could regulate the occurrence and development of desmoplasia.

### 1.3.3. Effect of NanoPue on Biodistribution of Second-Wave Injected Nanoparticles.

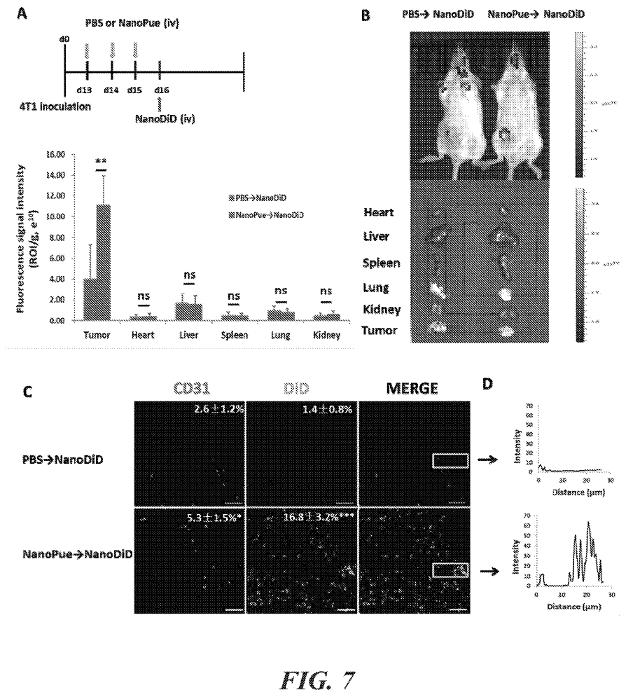

The stroma remodeling effect of the nanoPue treatment was further validated via the investigation of biodistribution of second-wave injected nanoparticles. The far-red fluorescence dye DiD was encapsulated in the same nanoemulsion formulation as in nanoPue. After 3 consecutive nanoPue or PBS treatments, 4T1 tumor-bearing mice were iv injected with DiD encapsulated testing nanoemulsion particles (nanoDiD) (FIG. 7A). In vivo and ex vivo imaging analyses demonstrated nearly 3-fold higher nanoDiD accumulation in the tumor of the animals pre-injected with nanoPue than with PBS (P<0.01) (FIG. 7B). No significant difference in the fluorescence signals of heart, liver, spleen, lung and kidney was observed between PBS and nanoPue group (FIG. 7B). These results indicated that pre-injection of nanoPue had changed TME and render them more permeable for the subsequently injected nanoparticles.

Frozen sections of tumors were stained for CD31 tumor vessel marker to observe the positional relationship between the nanoDiD and the blood vessels. In the mice pre-injected with PBS, not only did few nanoDiD appear in the tumor, their location was close to the vessels. Thus, these tumors in the PBS group showed limited nanoparticle extravasation. On the other hand, in the mice pre-injected with nanoPue, many more nanoDiD were found in the tumors (FIG. 7C). More importantly, these particles were located at positions far away from the vessels, as shown by ImageJ analysis of the data (FIG. 7D). The results indicate that pre-injection of nanoPue improves the penetration of nanoparticles in the tumor, allowing more nanoDiD to accumulate in the tumor and diffuse deeper into the parenchyma of the tumor. Thus, nanoPue significantly increased the enhanced permeability and retention (EPR) effect of the tumor. Fang et al., 2011; Maeda et al., 2000. The effect must be related to the fact that nanoPue could significantly reduce the fibrogenic status of the fibroblasts, increase vessel permeability and reduce the interstitial fluid pressure of the treated tumor. These profound changes of the tumor resulted from the action of a single drug formulated in a simple emulsion without any noticeable toxicity. The treated tumors were no longer desmoplastic; there was a significant decrease in the α-SMA positive fibroblasts and increase of DiD (FIG. 6).

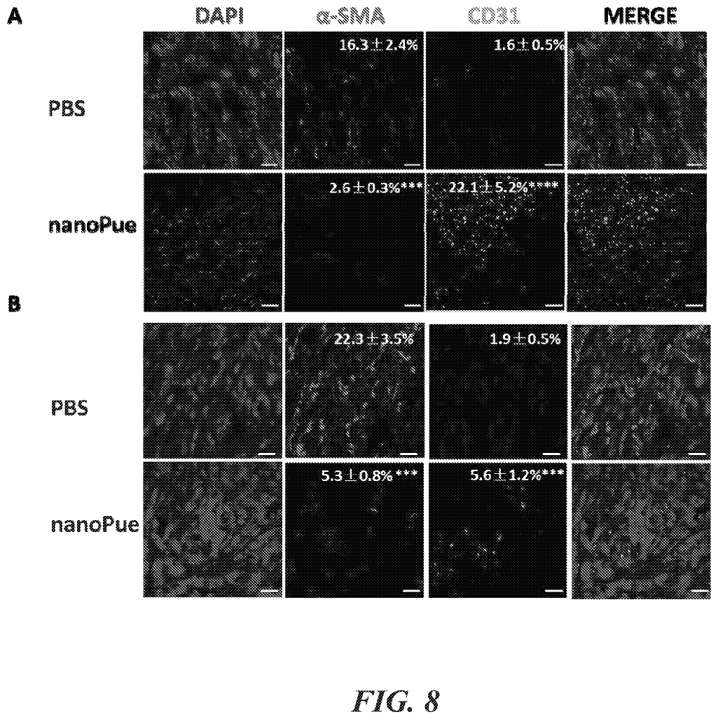

The effect of nanoPue was examined in another desmoplastic tumor, e.g., BPD6 melanoma. The number of CD31-labeled blood vessels in nanoPue treated group was significantly higher than that in the PBS treated group (P<0.0001). There also was a concomitant decrease in the α-SMA positive fibroblasts in the nanoPue treated group (P<0.001) (FIG. 8A). These observations were mirrored in the 4T1 tumors which were studied as a comparison (FIG. 8B). These results indicate that nanoPue could be a general reagent to reduce the desmoplasia of solid tumors. 1.3.4. Combination of NanoPue and PTX Polymer Therapy on 4T1 Tumor Model.

The chemotherapy treatment for TNBC is mainly based on PTX in the clinical applications since this cancer type is quite sensitive to PTX. Jean-Marc et al., 2016; Hu et al., 2015. The overall prognosis after PTX therapy, however, is still poor. Since nanoPue significantly increased the penetration of second-wave injected nanoparticles, without wishing to be bound to any one particular theory, it is thought that it can also facilitate the nano-formulated PTX chemotherapy in the highly desmoplastic TNBC. A PTX polymer nanoformulation (nanoPTX) (ZY Therapeutics

Inc.) was utilized to investigate the synergistic effect of nanoPue and PTX. The 4T1 tumor-bearing mice were administered three injections of nanoPue and then three injections of nanoPTX according to the treatment scheme illustrated in FIG. 9A. In comparison with PBS→PBS group, the PBS→nanoPTX and nanoPue→PBS groups showed a partial effect on tumor inhibition. Compared to the individual therapies, however, a combination therapy of nanoPue with nanoPTX significantly inhibited tumor growth (FIG. 9B). FIG. 9C shows that the average tumor weight at the endpoint in nanoPue→nanoPTX group was much lower than that in nanoPue→PBS group (P<0.001) and PBS→nanoPTX group (P<0.01). TUNEL assay demonstrated a large increase of apoptotic cells in nanoPue→nanoPTX combination treatment group compared to PBS→nanoPTX and nanoPue→PBS single treatment (FIG. 9D). Compared with the control and the single administration group, the expression of Ki67 protein (a marker for cell proliferation) in the tumor cells of the nanoPue→nanoPTX group was significantly down-regulated (p<0.0001) (FIG. 9E). The results showed that nanoPue and nanoPTX had a synergistic inhibitory effect on 4T1 tumor growth. Pre-injection of nanoPue could render the subsequent injection of nanoPTX polymer nanoformulation more effective in inhibiting tumors. The result was consistent with the in vivo imaging (FIG. 7B). Thus, nanoPue's activity in remodeling TME and alleviating tumor fibrosis was successfully translated to an improved efficacy of chemotherapy for the 4T1 tumor model.

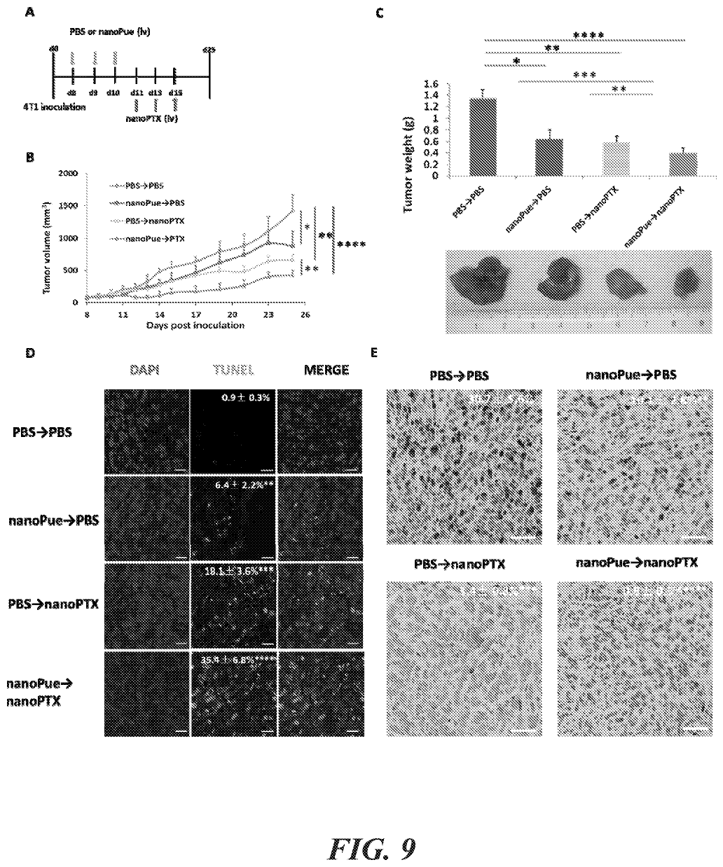

To investigate the toxic effects of combined drugs in mice, the body weight and serum chemistry of the treated mice were measured. The main organs of mice were examined by H&E staining method to evaluate any possible toxicity induced by the treatment. As shown in FIG. 10A, there were no significant differences in ALT, AST, BUN and creatinine between treatment group and PBS group, and all the parameters remained at normal levels. Interestingly, the weight of mice in PBS and nanoPue groups increased in varying degrees, while that of nanoPue→nanoPTX group remained unchanged and there was no significant difference among these three groups. The weight of mice in PBS→nanoPTX group, however, decreased significantly compared with other groups (FIG. 10B). The results showed that the chemotherapeutic drugs have a significant effect on the body weight of mice. Pre-injection of nanoPue could significantly reduce the toxicity of chemotherapeutic drugs. The results of H&E staining showed that no inflammation or necrosis in the heart, liver, spleen, kidney of nanoPue group compared with PBS group, indicating that the combined administration had no detectable toxicity in mice. Lung, however, seems to be the major metastatic site in the PBS and PBS→nanoPTX group. In the survival analysis, nanoPue→nanoPTX also showed significantly prolonged median survival compared with the PBS→PBS, PBS→nanoPue, and PBS→nanoPTX group

(FIG. 10C). The combination of nanoPue and nanoPTX not only shows a potent therapeutic effect but also achieves a long-lasting overall response. The combination of nanoPue and nanoPTX obviously suppressed metastasis in the lung (FIG. 10D).

### 1.3.5. NanoPue Induced 4T1 Tumor Immune Microenvironment Changes.

In the above experiments, the effects of pre-injection of nanoPue on tumor stromal microenvironment was systematically investigated (FIG. 5). Whether nanoPue had a favorable regulatory effect on tumor immune microenvironment after repeated nanoPue or PBS treatments in 4T1 breast cancer was further explored (FIG. 5A).

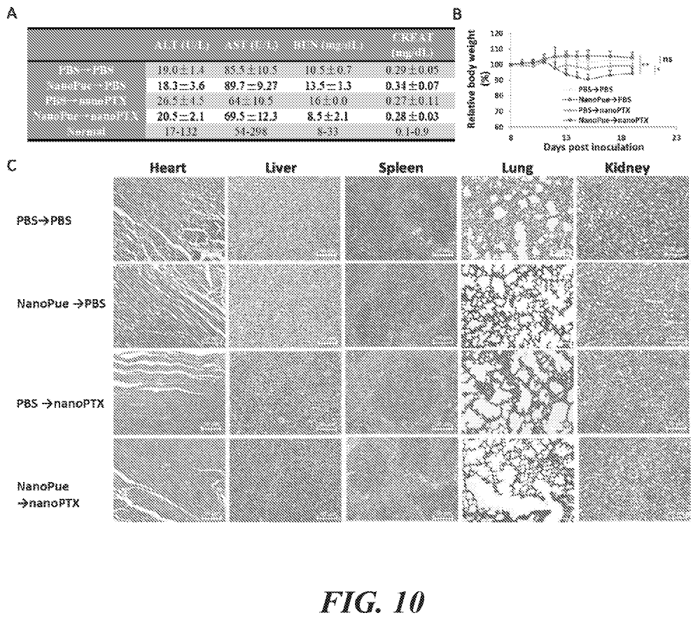

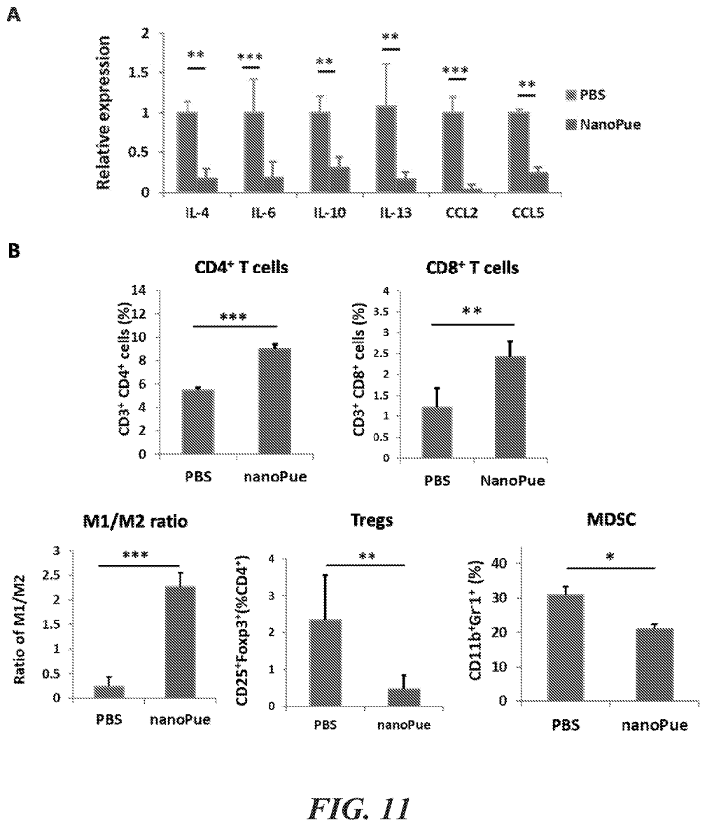

T cell, especially the CD8⁺ cytotoxic T cell-mediated cellular immunity is the main form of anti-tumor immunity. Han et al., 2018. Unfortunately, activated TAFs not only serve as a physical barrier for T cell penetration, but have evolved multiple immunoregulatory mechanisms via the secretion of immunosuppressive cytokines (e.g., IL-4, IL-6, IL-10, IL-13 and TGF-β) and expression of immune inhibitory molecules (e.g., PD-L1). Collectively, these immunosuppressive factors prevent T cell proliferation and differentiation, and trigger functional cytotoxic T cell death, thereby facilitating tumor cells to evade immune surveillance. Jiang et al., 2017. As expected, deactivation of TAFs by nanoPue significantly reduced intratumoral IL-4, IL-6, IL-10 and IL-13 (FIG. 11A), which led to increased infiltration of both CD8⁺ and CD4⁺ T cells into the tumor tissue (FIG. 11B). Noticeably, CD8⁺ T cells were more intensively promoted compared with CD4⁺ T cells (2.0 vs 1.6-fold increase) after the nanoPue treatment. In addition to directly impairing T cell function, activated TAFs are also responsible for the recruitment of circulating myeloid cells and regulatory T cells (Tregs) mediated by C-C motif chemokine 2 (CCL2) and C-C motif chemokine 5 (CCLS). Upon the stimulation by the suppressive T helper cell 2 (Th2) cytokines (e.g., IL-4, IL-10, and IL-13), these infiltrated myeloid cells differentiate into myeloid-derived suppressor cells (MDSCs) and M2 macrophages. Qian and Pollard, 2010. These recruited Tregs, MDSCs, and M2 macrophages have been reported to negatively correlate with the number of intratumoral CD8⁺ T cells and hence are associated with adverse prognosis of breast cancer. Denardo et al., 2009; Hao et al., 2012; Tan et al., 2009, Diaz-Montero et al., 2008; and Sinha et al., 2008. Therefore, the significant downregulation of CCL2 and CCLS together with the reduced intratumoral Th2 cytokine levels (FIG. 11A) further improved the immune microenvironment through reducing Tregs and MDSCs infiltration, as well as promoting M2 macrophage phenotype switch to pro-inflammatory M1 (FIG. 11B).

### 1.3.6. Combination of NanoPue and α-PD-L1 in 4T1 Tumor Model.

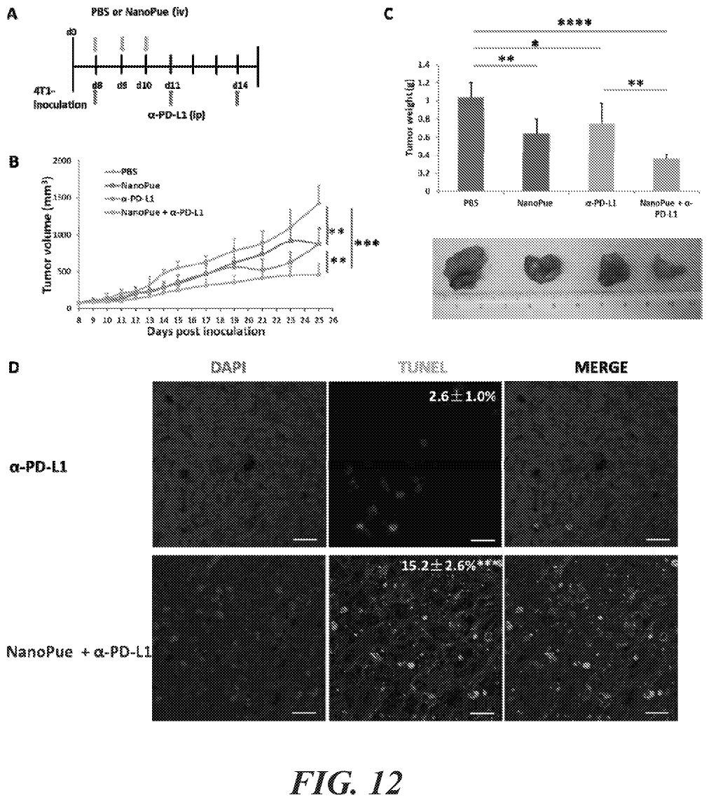

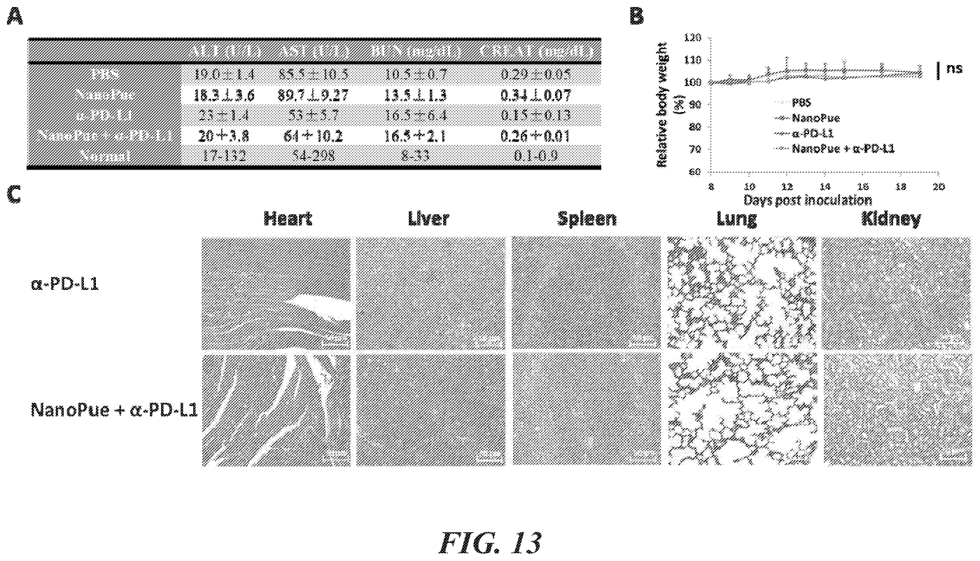

Although the PD-1/L1 antibody has achieved great success in the clinic, more than 80% of TNBC patients still fail to respond to the therapy. Hugo et al., 2016. Recent studies have demonstrated the correlation of better intratumoral T lymphocyte infiltration with higher patient response towards the PD-1/L1 antibody therapy. Champiat et al., 2016. Based on the presently disclosed findings that nanoPue can alleviate the immunosuppressive microenvironment of tumors, it was thought that nanoPue may enhance the activity of the checkpoint blockade immunotherapy. Accordingly, nanoPue was combined with α-PD-L1 in the treatment of 4T1 breast cancer (FIG. 12A). As shown in FIG. 12B, despite the moderate inhibition of tumor growth (P<0.01) by the α-PD-L1 or nanoPue monotherapy, the combined administration of nanoPue and α-PD-L1 dramatically slowed down the tumor progression (P<0.01) as also evidenced by the smallest tumor among all treatment groups 25 days after the tumor inoculation (FIG. 12C). TUNEL assay demonstrated significantly higher cytotoxic T cell-mediated apoptosis by the nanoPue +α-PD-L1 combination therapy than the α-PD-L1 monotherapy (P<0.01), further validating the indispensable role of nanoPue in improving the immunogenicity in tumors and synergizing α-PD-L1 to activate the T cell immune response (FIG. 12D). Compared with α-PD-L1 group, the expression of Ki67 protein in the tumor cells of the nanoPue→>α-PD-L1 group was significantly down-regulated (p<0.05) (FIG. 12E). No abnormal changes of serum ALT, AST, or BUN were observed 3 days after the last injection in each treatment group, suggesting high liver and kidney safety of the regimen. And no histological abnormity of heart, lung, spleen, and kidney was observed in any α-PD-L1 groups (FIG. 13). Further, in the survival analysis, the nanoPue→>α-PD-L1 group showed significantly prolonged median survival compared with the control and single treatment groups (FIG. 13D).

### 1.3.7. Summary

In the presently disclosed subject matter, an easy-to-scale-up nanoemulsion formulation was developed for the systemic delivery of puerarin with high EE and stability. This nanoPue formulation dramatically reduced the desmoplastic reaction in different types of solid tumors via downregulation of intratumoral ROS. The remodeled stromal microenvironment by the nanoPue treatment made better penetration of nanoparticles into the tumor parenchyma. Meanwhile, the nanoPue therapy significantly improved the tumor immune microenvironment and enhanced therapeutic efficiency of α-PD-L1 in a TNBC model. In summary, nanoPue, a robust TME modulator, could serve as an adjuvant therapy for both chemotherapeutic drugs and checkpoint blockade immunotherapies in highly desmoplastic tumors. Its relatively simple and scalable preparation also grants nanoPue a great potential for the clinical translation.

### 1.4 Materials and Methods

### 1.4.1 Materials.

Medium-chain triglyceride (Kollisolv® MCT 70) was purchased from BASF (Ludwigshafen Germany). Polyethylene glycol (15)-hydroxystearate (Kolliphor° HS15), glycerol and Fluorometric Hydrogen Peroxide Assay Kit were obtained from

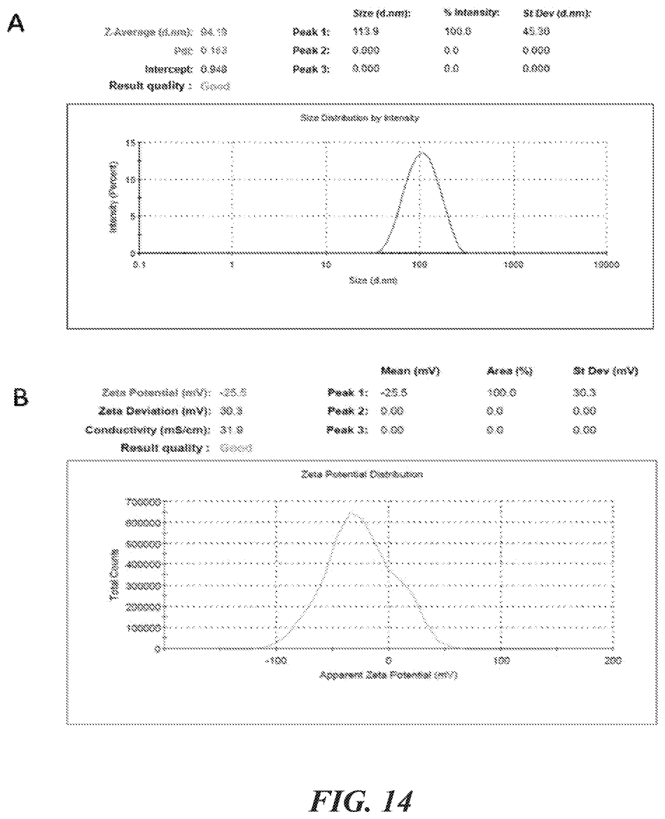

Sigma-Aldrich (St. Louis, Mo.). Puerarin and lecithin from soybean was obtained from TCI (Tokyo Kasei Kogyo, Japan). nanoPTX (ZY-010) was prepared by utilizing a biodegradable polysaccharide, which was provided by ZY Therapeutics Inc. (Research Triangle Park, N.C.). ZY-010 was a lyophilized dosage form of PTX nano-formulation which contained 10% of PTX and 90% of Dextran-Folic acid conjugated polymer. The details of the synthesis of the polymer and the preparation of PTX entrapped nanoformulation can be found in US Patent PCT/US18/28900 (Pharmaceutical Composition for in vivo Delivery, Method of Preparation of a Substantially Water-Insoluble Pharmacologically Active Agent for in vivo Delivery), which is incorporated herein by reference in its entirety. After reconstitution in saline solution, the particle size assessed by Dynamic Light Scattering was about 100 nm with a Particle Dispersion Index (PDI) of less than 0.2. Zeta-potential of the nano-formulation in saline solution was evaluated by the Zetasizer Nano ZS instrument (Malvern) and measured to be in the range −20 to −25 mV. The details of the particle size and Zeta-potential was listed in FIG. 14. DSPE-PEG-AEAA was synthesized in our laboratory. Miao et al., 2016; Jin et al., 2012.

### 1.4.2. Cell Lines, Animals and Antibodies.

Murine breast cancer 4T1 cells, mouse embryonic fibroblast cell line NIH3T3 and Murine BRAF mutant melanoma cell lines BPD6 were obtained from Tissue Culture Facility. 4T1 cells were stably transfected with the vector carrying the GFP, firefly luciferase, and the puromycin resistance gene. 4T1 cells and BPD6 cells were maintained in RPM-1640 media (Invitrogen) supplemented with FBS (10% v/v, Gibco), penicillin/streptomycin (1% v/v, Gibco), and puromycin (1 μg/mL, ThermoFisher) at 37° C. and 5% CO2 in a humidified atmosphere. NIH3T3 cells were cultured in Dulbecco's Modified Eagle's Media (DMEM) (Invitrogen, Carlsbad, Calif.), supplemented with FBS (10% v/v, Gibco) or 10% fetal bovine serum (Sigma, St. Louis Mo.), respectively, with penicillin (100 U/mL) (Invitrogen) and streptomycin (100 μg/mL) (Invitrogen).

Female Balb/C mice (8-10 weeks old) and female C57BL/6 mice (8-10 weeks old) were provided by Jackson Labs. All animal protocols were approved by the University of North Carolina at Chapel Hill's Institutional Animal Care and Use Committee.

Antibodies and primers used in the study for western blot, flow cytometry, and immunofluorescence staining are listed in Table 1 and 2.

TABLE 1
Antibodies Used in the Study

| Antibodies | Company | Catalog | Application |
|---|---|---|---|
| FITC Anti-CD8 | BioLegend | 100705 | flow cyt |
| Alexa Fluor ®594 | BioLegend | 100446 | flow cyt |
| Anti-CD4 | | | |
| Alexa Fluor ®488 | BioLegend | 101217 | flow cyt |
| Anti-CD11b | | | |
| APC-Cy7 | BioLegend | 101225 | flow cyt |
| Anti-CD11b | | | |
| PE-Cy7 | BioLegend | 100219 | flow cyt |
| Anti-CD3 | | | |
| APC Anti-Gr-1 | BioLegend | 108411 | flow cyt |
| PE Anti-CD206 | BioLegend | 141706 | flow cyt |
| Alexa Fluor ®594 | BioLegend | 123140 | flow cyt |
| Anti-F4/80 | | | |
| PerCp Anti-MHCII | BioLegend | 107623 | flow cyt |
| PE-Cy7 Anti-CD11c | BioLegend | 117317 | flow cyt |
| PE Anti-CD206 | BioLegend | 141706 | flow cyt |
| Alexa Fluor ®488 | BioLegend | 126406 | flow cyt |
| Anti-FoxP3 | | | |
| Anti-α-SMA | Abcam | ab184675 | flow cyt |
| GAPDH | Santa Cruz | I3015 | WB |
| Goat | Abcam | ab205718 | WB |
| anti-rabbit HRP | | | |
| Anti-NADPH | Abcam | ab133303 | WB |
| oxidase 4 | | | |
| Anti-Smad2 | Abcam | ab53100 | WB |
| (phospho S467) | | | |
| Anti-Smad3 | Abcam | ab63403 | WB |
| (phospho S213) | | | |
| Anti-HIF-1α | Cell | mAb #36169 | WB |
| | signaling | | |
| Anti-α-SMA | Abcam | ab5694 | IF |
| Anti-CD31 | BioLegend | 102432 | IF |

IF: immunofluorescence.
IHC: immunohistochemistry.
Flow cyt: Flow cytometry.

TABLE 2
Primers used in this study

| Primer | Applied Biosystems |
|---|---|
| Mouse GAPDH | Mm99999915_g1 |
| Mouse TGF-β | Mm01178820_m1 |
| Mouse IL4 | Mm00445259_m1 |
| Mouse IL6 | Mm00446190_m1 |
| Mouse IL10 | Mm01288386_m1 |
| Mouse IL13 | Mm00434204_m1 |
| Mouse CCL2 | Mm00441242_m1 |
| Mouse CCL5 | Mm01302428_m1 |
| Mouse PDGF-B | Mm00440674_m1 |
| Mouse TNF-α | Mm00443260_g1 |
| Mouse FGF-2 | Mm01285715_m1 |

All the primers were provided by ThermoFisher Scientific.

### 1.4.3. Screening of TCMs for ROS Inhibition.

Based on literature and Chinese medicine pharmacology, 10 anti-fibrosis TCMs were selected to screen the ROS inhibition effects on TGF-β activated NIH3T3 cells. NIH3T3 cells were pre-stimulated with 10 ng/mL TGF-β for 24 h and 2×10³ cells/well were seeded in a 96-well black plate. Then, the cells were treated with different TCMs. Cells were also treated with different concentration of puerarin and nanoPue as shown in FIG. 2A. After these drugs were added, the cells were further cultured for 48 h. Then the concentration of ROS was determined with the Fluorometric Hydrogen Peroxide Assay Kit according to the manufacturer's instructions. The fluorescence intensity measured by using a fluorescence microplate reader (FLx800, Biotek Instrument Inc., Winooski, VT, USA) at excitation and emission wavelengths of 485 and 530 nm, respectively.

1.4.4. Cytotoxicity of Puerarin. The addition of puerarin and nanoPue to NIH3T3 cells was the same as the ROS assay. After 48 h of incubation, the culture medium was discarded and the cell layers were washed twice with PBS. Then 100 μL culture medium and 10 μL MTT solution (5 mg/mL) were added and incubated for 4 h at 37° C. After the supernatant was removed, 150 μL DMSO was added and OD value was determined at 492 nm by using a multidetection microplate reader (Plate CHAMELEONTM V-Hidex). The cell viability rate of each concentration was calculated according to formula (1).

Cell viability %=(OD_{dosing group}/OD_{control group})×100% (1)

1.4.5. Preparation and Characterization of NanoPue. The aqueous phase was prepared by weighing an amount of glycerol (0.3 g) into 6 mL glucose solution (5%, w/w). DSPE-PEG-AEAA (1.5 mg) was added to the above mixture. The oil phase was prepared by dispersing 1.2 mg puerarin, 15% (w/w) of lecithin from soybean, 30% (w/w) Kolliphor° HS15 in MCT 70. The water phase was added into the oil phase quickly. The mixture was well mixed by a PC-351 hot plate-stirrer and incubated at 50° C. for 20 min. The crude emulsion was sonicated by using a Fisher Scientific sonic dismembrator model 100 at 600 w for 10 min. The emulsion was extruded through 0.22-μm polycarbonate membranes. NanoDiD was prepared by using the same method except that 2 mg of DiD was added to the oil phase. The particle size and zeta potential measurements were conducted with the Zetasizer (Nano ZS, Malvern Instruments Ltd., UK). The emulsion was negatively stained with 2% uranyl acetate and the emulsion morphology was observed by JEOL 100 CX II TEM (JEOL, Japan). The EE of nanoPue was measured by using the mini-column (Sephadex G-50) centrifugation method. The puerarin concentration was analyzed by a high-performance liquid chromatography (HPLC, Agilent LC1100) at the wavelength of 250 nm.

### 1.4.6. Stability of NanoPue In Vitro.

To investigate the placement stability of nanoemulsion, nanoPue and puerarin suspension were incubated at 4° C. for different time. The change of particle size and EE% of samples were then measured by using the above methods separately. The release of puerarin from emulsion was determined by using a dynamic dialysis method. A total of 1 mL nanoPue and puerarin suspension were loaded in a dialysis bag and incubated in 100 mL of PBS at pH 7.4 at 37° C. At different times, 5 mL release medium was taken out and determined by using HPLC (Agilent LC1100) at the wavelength of 250 nm. The cumulative release of puerarin was calculated by the measured values of each time.

### 1.4.7. Pharmacokinetic Study.

Mice were injected via the tail vein a single dose of nanoPue and suspension respectively at a dose of 35 mg/kg puerarin. Blood samples were collected via eye puncture at different time after administration. The blood samples were centrifuged at 5,000×g for 10 min and stored in a freezer at −20° C. Plasma samples (50 μL) were added 150 μL of methanol-acetonitrile (1:1, v/v). After vortex for 1 min, the mixture was centrifuged at 5,000×g for 10 min and the supernatant was analyzed by HPLC. HPLC conditions were the same as the method mentioned in the determination of EE.

The concentration-time data of puerarin was processed by the 3P97 pharmacokinetic calculation program to calculate the pharmacokinetic parameters.

### 1.4.8. Establishment of Tumor Model in Mice.

4T1 and BPD6 tumor models were established in Balb/C and C57BL/6 female mice, respectively. 4T1 cells and BPD6 cells were harvested and washed in PBS (pH 7.4). For 4T1 tumor model, 1×10⁶ cells suspension was injected into the mammary fat pads of the mice. For BPD6 tumor model, 1×10⁶ BPD6 cells were injected into subcutaneous tissue in the lower flank area of Balb/C mice. The growth of 4T1 and BPD6 tumors were followed by directly measuring the tumor size by using a caliper.

### 1.4.9. Safety Evaluation of NanoPue.

At the end of the endpoint, the mice were sacrificed and whole blood was obtained and centrifuged at 8,000×g for 10 min to collect serum. ALT, AST, BUN, and creatinine, levels were determined as indicators of hepatic and renal damage. The body weights of mice were measured every other day from the beginning of treatment. The organs of mice, such as heart, liver, spleen, lung and kidney were fixed with 4% paraformaldehyde (PFA) and then were soaked in 70% ethanol overnight. H&E staining of organs was operated by UNC histology facility and the slides were observed by using fluorescence microscopy (Nikon, Tokyo, Japan) with 20x objective.

### 1.4.10. Effect of NanoPue on TME.

Mice bearing 4T1 tumors were randomized blindly into 3 treatment groups (n=7): Untreated group (PBS), blank emulsion and nanoPue group (35 mg/kg). At the end of the endpoint, the mice were sacrificed and tumors were collected and weighed, then for H&E staining, Masson's trichrome staining, immunofluorescence staining, Western Blot Analysis, flow cytometry analysis and RT-PCR assay.

### 1.4.11. Immunofluorescence Staining and Masson's Trichrome Staining.

The tumor tissues were taken out from the mice and soaked in 4% PFA, 15% and 30% sucrose solutions for 24 h at 4° C., respectively. The tumors were embedded in optimal cutting temperature embedding medium (Fisher Scientific) and cut into 10 μm sheets by Leica CM1850 cryostat (Germany). The slides were washed 3 times by 1×PBS, permeabilized with 1% Triton and blocked by 5% goat serum. The primary antibodies with or without fluorescence were incubated with the slides 24 h at 4° C. It is necessary that the slides were incubated with fluorescent secondary antibodies if the primary antibody does not have fluorescence. Finally, the Nuclei were stained with DAPI. The slides were observed by using laser scanning confocal microscope (Zeiss, LSM 710).

The Masson's trichrome assay was performed to investigate collagen among tumor tissues. Tumor tissues were fixed with 4% PFA and then were soaked in 70% ethanol overnight. The slides were stained by using a Masson's trichrome Kit by the UNC Tissue Procurement Core. The slides were observed by using fluorescence microscopy (Nikon, Tokyo, Japan). A minimum of five randomly selected microscopic fields were quantitatively analyzed by using ImageJ software.

### 1.4.12. Western Blot Analysis.

4T1 tumor bearing mice received six iv injection of nanoPue and PBS were sacrificed and the tumor tissues were collected. A 50-mg tumor sample was homogenized and lysed by using 500 μL radioimmunoprecipitation assay (RIPA) buffer containing 1% protease inhibitor (protease inhibitor cocktail and phosphatase inhibitor cocktail). The total protein concentration was determined by Pierce™ BCA

Protein Assay Kit (Thermo Scientific, USA). Subsequently, 25 μg protein was loaded for Western Blot Analysis. The primary antibodies for HIF-la, α-SMA, p-SMAD2, p-SMAD3, NOX 4 were used for in vivo western blot analysis and GAPDH was used as a control.

### 1.4.13. Flow Cytometry.

The tumor tissues were placed on ice and incubated with collagenase A buffer and DNAase at 37° C. for 40 min. To obtain single cell suspension, FACS buffer (PBS containing 3% serum) was added and ground with filter. The mixture was centrifuged at 1,200×g for 10 min. One mL ammonium-chloride-potassium buffer and 10 mL FACS were added to the sediment. Then the cell concentration was adjusted to 1-3×10⁶ cells/mL. According to the protocol of manufacturer, cells are stained by antibodies on the surface or intracellular. The cells were fixed by adding 4% PFA and stored at 4° C. and analyzed via LSRFortessa (BD Biosciences).

### 1.4.14. Quantitative Real-Time PCR (RT-PCR) Assay.

20-30 mg tumor tissue was placed on ice and added 600 μL RNeasy lysis buffer, then broke by using the tissue tearor (Biospec products, Inc., USA). The total RNA was obtained from the tumor tissue homogenate followed the method of the RNeasy microarray tissue mini kit (Qiagen, Hilden, Germany). Subsequently RNA was reverse transcribed into cDNA by using the iScriptTM cDNA Synthesis Kit (BIO-RAD). The concentration of RNA and cDNA were both determined by using NanoDropTM 2000 Spectrophotometers (ThermoFisher scientific, USA). The obtained cDNA was amplified by using the TaqManTM Gene Expression Master Mix. RT-PCR was performed by using the 7500 Real-Time PCR System and data were analyzed with the 7500 Software. The GAPDH RNA expression was used as normalized control.

### 1.4.15. Effect of Repeated Injection of NanoPue on the Distribution of Subsequently Injected NanoDiD.

After 3 injections of nanoPue (35 mg/kg, everyday), the mice were administered with nanoDiD at the DiD dose of 0.75 mg/kg. Twenty-four h later, the mice were imaged by using the IVIS® Kinetics Optical System (Perkin Elmer, CA) at excitation and emission wavelengths of 640 and 670nm, respectively. Then the mice were sacrificed and major organs and tumors were collected. The bio-distribution of nanoDiD was quantitatively visualized with IVIS system also.

1.4.16. Effect of NanoPue Combined with Chemotherapeutic Drugs on Tumor Inhibition.

Mice bearing 4T1 tumors were randomized blindly into four treatment groups (n=5): PBS→PBS group, nanoPue→PBS group, PBS→nanoPTX group and nanoPue→nanoPTX group and the treatment scheme is shown in FIG. 9A. The tumor volumes were measured by using a vernier caliper and calculated through the following equations:

Vt=(a×b²)/2 (2)

where a and b represent the long and short axis, respectively. At the end of the endpoint, the mice were sacrificed and the tumors were collected and weighed, then H&E staining and TUNEL assay were performed.

### 1.4.17. TUNEL Assay.

Apoptosis experiments were carried out by using a TUNEL assay kit (DeadEnd™ Fluorometric TUNEL System, Promega) following the manufacturer's protocol. Genomic fragmented cells were stained with green fluorescence of FITC and defined as TUNEL-positive nuclei. Then nuclei were stained with DAPI (ThermoFisher Scientific, USA). The images were taken by using laser scanning confocal microscope (Zeiss, LSM 710). A minimum of 5 randomly selected microscopic fields were quantitatively analyzed by using ImageJ software.

### 1.4.18. Statistical Analysis.

Quantitative results were expressed as mean ±SD. The analysis of variance was completed using a one-way ANOVA or a two-tailed Student's t test. A P value less than 0.05 was considered statistically significant.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Bremnes, M. R.; Donnem, T.; Al-Siad, S.; Al-Shibli, K.; Andersen, S.; Sirera R.; Camps, C.; Marinez, I.; Busund, L-T. The role of tumor stroma in cancer progression and prognosis: emphasis on carcinoma-associated fibroblasts and non-small cell lung cancer. J Thorac Oncol 2011, 6, 209-217.

Valkenburg, K. C.; Groot, A. E. D.; Pienta, K. J. Targeting the tumour stroma to improve cancer therapy. Nat Rev Clin Oncol 2018, 15, 366.

Zhang, B.; Jiang, T.; Shen, S.; She, X. J.; Tuo, Y. Y.; Hu, Y.; Pang, Z. Q.; Jiang, X. G. Cyclopamine disrupts tumor extracellular matrix and improves the distribution and efficacy of nanotherapeutics in pancreatic cancer. Biomaterials 2016, 103, 12-21.

Mariathasan, S.; Turley, S.J.; Nickles, D.; Castiglioni, A.; Yuen, K.; Y. I.; Kadel, E. E.; Koeppen, H.; Astarita, J. L.; Cubas, R.; Jhunjhunwala, S.; Banchereau, R.; Yang, Y. G.; Guan, Y. G.; Chalouni, C.; Ziai, J.; Senbabaoglu, Y.; Santoro, S.; Sheinson, D.; Hung, J.; Giltnane, J. M.; Pierce, A. A.; Mesh, K.; Lianoglou, S.; Riegler, J.; Carano, R. A, D.; Eriksson, P.; HOglund, M.; Somarriha, L.; Halligan, D. L.; van der Heijden, M. S.; Loriot, Y.; Rosenberg, J. E.; Fong, L.; Mellman, I.; Chen, D. S.; Green, M.; Derleth, C.; Fine, G. D.; Hegde, P. S.; Bourgon, R.; Powles, T. TGFβ attenuates tumour response to PD-L1 blockade by contributing to exclusion of T cells. Nature 2018, 544, 544.

Wiesner, T.; Kiuru, M.; Scott, S. N.; Arcila, M.; Halpern, A. C.; Hamann, T.; Berger, M. F.; Busam, K. J. NF1 mutations are common in desmoplastic melanoma. Am J Surg Pathol 2015, 39, 1357.

Zhao, X.D.; Subramanian, S. Intrinsic resistance of solid tumors to immune checkpoint blockade therapy. Cancer Res 2017, 77, 817-822.

Carey, L. A.; Winer, E.; Viale, G.; Cameron, D.; Gianni, L. Triple-negative breast cancer: disease entity or title of convenience? Nat Rev Clin Oncol 2010, 7, 683.

Denkert, C.; Liedtke, C.; Tutt, A.; Minckwitz, G. V. Molecular alterations in triple-negative breast cancer-the road to new treatment strategies. The Lancet 2017, 389, 2430-2442.

Miao, L.; Wang, Y.; Lin, C. M.; Xiong, Y.; Chen, N.; Zhang, L.; Kim, W. Y.; Huang, L. Nanoparticle modulation of the tumor microenvironment enhances therapeutic efficacy of cisplatin. J Control Release 2015, 217, 27-41.

Arcucci, A.; Ruocco, M. R.; Granato, G.; Sacco, A. M.; Montagnani, S. Cancer: an oxidative crosstalk between solid tumor cells and cancer associated fibroblasts. Biomed Res Int 2016, 1-7.

Yang, Y. H.; Bazhin, A. V.; Werner, J.; Karakhanova, S. Reactive oxygen species in the immune system. Int Rev Immunol 2013, 32, 249-270.

Bacanli, M.; Aydi., S.; Bsaran, A. A.; Basaran., N. A Phytoestrogen Puerarin and Its Health Effects. In: Polyphenols: Prevention and Treatment of Human Disease. Academic Press 2018. P. 425-431.

Wei, S. Y.; Chen, Y.; Xu, X.Y. Progress on the pharmacological research of puerarin: a review. Chin J Nat Med 2014, 12, 407-414.

Hou, Y-X.; Zhang, H.; Peng, C. Puerarin: a review of pharmacological effects. Phytother Res 2014, 28, 961-975.

Meitzler, J. L.; Antony, S.; Wu, Y. Z.; Juhasz, A.; Liu, H.; Jiang, G. J.; Lu, J. M.; Roy, K.; Doroshow, J. H. NADPH oxidases: a perspective on reactive oxygen species production in tumor biology. Antioxid Redox Sign 2014, 20, 2873-2889.

Son, B.; Kwon, T.; Lee, S.; Han, I.; Kim, W.; Youn, H.; Youn, B. CYP2E1 regulates the development of radiation-induced pulmonary fibrosis via ER stress-and ROS-dependent mechanisms. Am J Physiol-Lung C 2017, 313, L916-L929.

Wei, D.; Zhang, X. Solubility of puerarin in the binary system of methanol and acetic acid solvent mixtures. Fluid Phase Equilibr 2013, 339, 67-71.

Quan, D. Q.; Xu, G. X. Formulation optimization of self-emulsifying preparations of puerarin through self-emulsifying performances evaluation in vitro and pharmacokinetic studies in vivo. Acta pharm Sin 2007, 42, 886-891.

Chung, M. J.; Sung, N-J.; Park, C-S.; Kweon, D-K.; Mantovani, A-B.; Moon, T-W.; Lee, S-J.; Park, K-H. Antioxidative and hypocholesterolemic activities of water-soluble puerarin glycosides in HepG2 cells and in C57 BL/6J mice. Eur J Pharmacol 2008, 578, 159-170.

Banerjee, R.; Tyagi, P.; Li, S.; Huang, L. Anisamide-targeted stealth liposomes: A potent carrier for targeting doxorubicin to human prostate cancer cells. Inter J Cancer 2004, 112, 693-700.

Goodwin, T. J; Huang, L. On the article “Findings questioning the involvement of Sigma-1 receptor in the uptake of anisamide-decorated particles” [J. Control. Release 224 (2016) 229-238] Letter to the Editor 1 (Sep. 14, 2016). J Control Release 2016, 243,382-385.

van Waarde. A.; Rybczynska, A. A.; Ramakrishnan, N. K.; Ishiwata, K.; Elsinga, P.H.; Dierckx , R.A.; Potential applications for sigma receptor ligands in cancer diagnosis and therapy. Biochim Biophys Acta 2015, 1848, 2703-2714.

Miao, L.; Newby, J. M.; Lin, C. M.; Zhang, L.; Xu, F.; Kim, W. Y.; Forest, M. G.; Lai, S. K.; Milowsky, M. I.; Wobker, S. E.; Huang, L. The binding site barrier elicited by tumor-associated fibroblasts interferes disposition of nanoparticles in stroma-vessel type tumors. ACS nano 2016, 10, 9243-9258.

Jin, S. E.; Son, Y. K.; Min, B-S.; Jung, H. A.; Choi, J. S. Anti-in-flammatory and antioxidant activities of constituents isolated fromPueraria lobata roots. Arch Pharm Res Vol 2012, 35, 823-837.

Liu, C. M; Zheng, G. H.; Ming, Q. L.; Sun, J. M.; Cheng, C. Protective effect of puerarin on lead-induced mouse cognitive impairment via altering activities of acetyl cholinesterase, monoamine oxidase and nitric oxide synthase. Environ Toxicol Ph 2013, 35, 502-510.

Zhang, S. H.; Ji, G.; Liu, J. W. Reversal of chemical-induced liver fibrosis in Wistar rats by puerarin. J. Nutr Biochem 2006, 17, 485-491.

Clément, S.; Hinz, B.; Dugina, V.; Gabbiani, G.; Chaponnier, C. The N-Terminal Ac-EEED Sequence Plays a Role in α-Smooth Muscle Actin Incorporation into Stress Fibers. J Cell Sci 2015, 13, 1395-1404.

Kato, M.; Zhang, J.; Wang, M.; Lanting, L.; Yuan, H.; Rossi, J. J.; Natarajan, R. MicroRNA-192 in diabetic kidney glomeruli and its function in TGF-β-induced collagen expression via inhibition of E-box repressors. P Natl Acad Sci 2007, 104, 3432-3437.

de Souza Oliveira, L. S.; Araújo, A. A.; Junior, R. F. A.; Barboza, C. A. G.; Borges, B. C. D.; Silva, J. S. P. Low-level laser therapy (780 nm) combined with collagen sponge scaffold promotes repair of rat cranial critical-size defects and increases TGF-β, FGF-2, OPG/RANK and osteocalcin expression. Int J Exp. Patho 2017, 2, 75-85.

Selman, M.; Thannickal, V. J.; Pardo, A.; Zisman, D. A.; Martinez, F. J.; Lynch Iii, J. P. Idiopathic pulmonary fibrosis: pathogenesis and therapeutic approaches. Drugs 2004, 64, 405-431.

Samarakoon, R.; Overstreet, J. M.; Higgins, P. J. TGF-β signaling in tissue fibrosis: redox controls, target genes and therapeutic opportunities. Cell Signal 2013, 25, 264-268.

Michaeloudes, C.; Sukkar, M. B.; Khorasani, N. M.; Bhaysar, P. K.; Chung, K. F. TGF-β regulates Nox4, MnSOD and catalase expression, and IL-6 release in airway smooth muscle cells. Am J Physiol-Lung C 2010, 300, L295-L304.

Veith, C.; Hristova, M.; Boots, A.; van Schooten, F. J.; van der Vliet, A. LSC-2017-Profibrotic signaling by TGF-β involves NADPH oxidase 4 dependent activation of tyrosine kinase Src and mitochondrial ROS. Eur Respir J 2017, 50.

Chan, E. C.; Peshavariya, H. M.; Liu, G. S.; Jiang, F.; Lim, S. Y.; Dusting, G. J. Nox4 modulates collagen production stimulated by transforming growth factor (β1 in vivo and in vitro. Biochem Bioph Res Co 2013, 430, 918-925.

Feng, H. L.; Wang, J.; Chen, W.; Shan, B. E.; Guo, Y.; Xu, J. F.; Wang, L.; Guo, P.; Zhang, Y. Z. Hypoxia-induced autophagy as an additional mechanism in human osteosarcoma radioresistance. J Bone Oncol 2016, 5, 67-73.

Garrido-Urbani, S.; Jaquet, V.; Imhof, B. A. ROS and NADPH oxidase: key regulators of tumor vascularisation. Med Sci (Paris) 2014, 30, 415-421.

Fang, J.; Nakamura, H.; Maeda, H. The EPR effect: unique features of tumor blood vessels for drug delivery, factors involved, and limitations and augmentation of the effect. Adv Drug Deliver Rev 2011, 63, 136-151.

Maeda, H.; Wu, J.; Sawa, T.; Matsumura, Y.; Hori, K. Tumor vascular permeability and the EPR effect in macromolecular therapeutics: a review. J Control Release 2000, 65, 271-284.

Jean-Marc, F.; Anne-Claire, H. B.; Olivier, C.; Alain, L.; Bruno, S.; Philippe,

F.; Robert, H.; Mathilde, D.; Jerome, D.; Mustapha, A.; Remy, L. Weekly paclitaxel, capecitabine, and bevacizumab with maintenance capecitabine and bevacizumab as first-line therapy for triple-negative, metastatic, or locally advanced breast cancer: Results from the GINECO A-TaXel phase 2 study. Cancer 2016, 122, 3119-3126.

Hu, X. C.; Zhang, J.; Xu, B. H.; Cai, L.; Ragaz, J.; Wang, Z. H.; Wang, B. Y.; Teng, Y. E.; Tong, Z. S.; Pan, Y. Y.; Yin, Y. M.; Wu, C. P.; Jiang, Z. F. Wang, X. J.; Lou, G. Y.;. Liu, D. G ; Feng, J. F.; Luo, J. F.; Sun, K.; Gu, Y. J.; Wu, J.; Shao, Z. M. Cisplatin plus gemcitabine versus paclitaxel plus gemcitabine as first-line therapy for metastatic triple-negative breast cancer (CBCSG006): a randomized, openlabel, multicenter, phase 3 trial. Lancet Oncol 2015, 16, 436-446.

Han, C. Y.; Byoung, S. K. Chimeric antigen receptor T-cell therapy for cancer: a basic research-oriented perspective. Immunotherapy 2018, 10, 221-234.

Jiang, H.; Hegde, S.; DeNardo, D. G. Tumor-associated fibrosis as a regulator of tumor immunity and response to immunotherapy. Cancer Immunol Immun 2017, 66, 1037-1048.

Qian, B. Z.; Pollard, J. W. Macrophage diversity enhances tumor progression and metastasis. Cell 2010, 141, 39-51.

Denardo, D. G.; Barreto, J. B.; Andreu, P.; Vasquez, L.; Tawfik, D.; Kolhatkar, N.; Coussens, L. M. CD4+ T cells regulate pulmonary metastasis of mammary carcinomas by enhancing protumor properties of macrophages. Cancer Cell 2009, 16, 91-102.

Hao, N. B. ; LU, M. H.; Fan, Y. H.; Cao, Y. L.; Zhang, Z. R.; Yang, S. M. Macrophages in tumor microenvironments and the progression of tumors. Clin Dev Immunol 2012, 1-11.

Tan, M. C. B.; Goedegebuure, P. S.; Belt, B. A.; Flaherty, B.; Sankpal, N.; Gillanders, W. E.; Eberlein, T. J.; Hsieh, C. S.; Linehan, D.C. Disruption of CCRS-dependent homing ofregulatory T cells inhibits tumor growth in a murine model of pancreatic cancer. J Immunol 2009, 182, 1746-1755.

Diaz-Montero, C. M.; Salem, M. L.; Nishimura, M. I.; Garrett-Mayer, E.; Cole, D. J.; Montero, A. J. Cancer Immunol Immun 2008, 58, 49.

Sinha, P.; Okoro, C.; Foell, D.; Freeze, H. H.; Ostand-Rosenberg, S.; Srikrishna, G. Proinflammatory S100 proteins regulate the accumulation of myeloid-derived suppressor cells. J Immunol 2008, 181, 4666-4675.

Hugo, W.; Zaretsky, J. M.; Sun, L.; Johnson, D. B.; Ribas, A.; Lo, R. S. Genomic and Transcriptomic Features of Response to Anti-PD-1 Therapy in Metastatic Melanoma. Cell 2016, 165, 35-44.

Champiat, S.; Dercle, L.; Amman, S.; Massard, C.; Hollebeaque, A.; Postel-Vinay, S.; Chaput, N.; Eggermont, A.; Marabelle, A.; Soria, J. C.; Ferte, C. Hyperprogressive disease (HPD) is a new pattern of progression in cancer patients treated by anti-PD-1/PD-L1. Clin Cancer Res 2016, 23, 1920.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A nanoemulsion comprising puerarin, or a derivative thereof, for use in treating cancer.

2. The nanoemulsion of claim 1, wherein the nanoemulsion comprises lecithin.

3. The nanoemulsion of claim 1, further comprising a targeting ligand.

4. The nanoemulsion of claim 3, wherein the targeting ligand is aminoethylanisamide (AEAA).

5. The nanoemulsion of claim 1, wherein the nanoemulsion comprises spherical particles.

6. The nanoemulsion of claim 5, wherein the spherical particles have a diameter of about 112±5 nm.

7. The nanoemulsion of claim 1, wherein the nanoemulsion has a zeta potential of about −5.3±0.6 mV.

8. The nanoemulsion of claim 1, wherein the nanoemulsion has an encapsulation efficiency of about 82.4±3.2% for puerarin.

9. A method for treating a cancer in a subject in need of treatment thereof, the method comprising administering a therapeutically effective amount of a nanoemulsion of any of claims 1-8 to the subject to treat the cancer.

10. The method of claim 9, wherein the method further comprises treatment with one or more therapeutic agents in combination with the nanoemulsion of any of claims 1-8.

11. The method of claim 10, wherein the one or more therapeutic agents comprises one or more chemotherapeutic agents.

12. The method of claim 11, wherein the one or more chemotherapeutic agents comprises paclitaxel.

13. The method of claim 12, wherein the paclitaxel comprises a polymer nanoformulation of paclitaxel.

14. The method of claim 9, further comprising a PD-L1 blockade therapy.

15. The method of claim 14, wherein the PD-L1 blockade therapy comprises administering α-PD-L1 to the subject in combination with the nanoemulsion of any of claims 1-8.

16. The method of any of claims 9-15, wherein the treating of the cancer reduces metastasis of the cancer.

17. The method of any of claims 9-15, wherein the treating of the cancer decreases a weight of a tumor comprising the cancer.

18. The method of any of claims 9-15, wherein the treating of the cancer inhibits growth of a tumor comprising the cancer.

19. The method of any of claims 9-15, wherein the treating of the cancer includes a remodeling of a microenvironment of a tumor comprising the cancer.

20. The method of any of claims 9-15, wherein the treating of the cancer includes deactivating one or more tumor associated fibroblasts (TAFs).

21. The method of any of claims 9-15, wherein the treating of the cancer includes a reduction of α-SMA positive TAFs in one or more tumors comprising the cancer and/or a inhibiting expression of α-SMA in one or more tumors comprising the cancer.

22. The method of any of claims 9-15, wherein the treating of the cancer includes a reduction of intratumoral IL-4, IL-6, IL-10 and IL-13.

23. The method of any of claims 9-15, wherein the treating of the cancer includes increasing infiltration of CD8⁺ and CD4⁺ T cells into a tumor of the cancer.

24. The method of any of claims 9-15, wherein the treating of the cancer includes one or more of downregulation of CCL2 and CCLS, reducing intratumoral Th2 cytokine levels, reducing Tregs and MDSCs infiltration, promoting M2 macrophage phenotype switch to pro-inflammatory M1, and combinations thereof.

25. The method of any of claims 9-15, wherein the treating of the cancer includes downregulation of reactive oxygen species (ROS) production in an activated myofibroblast.

26. The method of any of claims 9-15, wherein the treating of the cancer reduces deposition of collagen in the extracellular matrix (ECM).

27. The method of any of claims 9-15, wherein the treating of the cancer alleviates desmoplasia.

28. The method of any of claims 9-15, wherein the treating of the cancer inhibits one or more profibrogenic cytokines.

29. The method of claim 28, wherein the one or more profibrogenic cytokines are selected from the group consisting of transforming growth factor-0 (TGF-(β), fibroblast growth factor (FGF-2), platelet-derived growth factor B (PDGF-B), and tumor necrosis factor (TNF-α).

30. The method of any of claims 9-15, wherein the treating of the cancer downregulated the expression of NOX4, HIF-1α, α-SMA, p-SMAD2 and p-SMAD3 in a tumor comprising the cancer.

31. The method of any of claims 9-15, wherein the treating of the cancer includes increasing an enhanced permeability and retention (EPR) effect of a tumor comprising the cancer.

32. The method of any of claims 9-15, wherein increasing the enhanced permeability and retention (EPR) effect of a tumor comprising the cancer includes reducing a fibrogenic status of one or more fibroblasts, increasing vessel permeability, and reducing an interstitial fluid pressure of a tumor comprising the cancer.

33. The method of any of claims 9-32, wherein the cancer is selected from breast cancer and melanoma.

34. The method of claim 33, wherein the breast cancer comprises triple negative breast cancer.
